(19) United States
(12) Patent Application Publication
(10) Pub. No.: US 2022/0249612 A1
(43) Pub. Date: Aug. 11, 2022

(54) COMPOSITIONS FOR TREATMENT OF AZOOSPERMIA, METHODS FOR PREPARING THE SAME AND APPLICATIONS THEREOF

(71) Applicants: Vasanthi PALANIVEL, Bangalore (IN); Shrinivas RANGACHARI, Chennai (IN)

(72) Inventors: Vasanthi PALANIVEL, Bangalore (IN); Shrinivas RANGACHARI, Chennai (IN)

(21) Appl. No.: 17/626,882

(22) PCT Filed: Jul. 13, 2020
(86) PCT No.: PCT/IB2020/056582

§ 371 Date: Jan. 13, 2022

(30) Foreign Application Priority Data
Jul. 15, 2019 (IN) 201941028254

(51) Int. Cl. A61K 38/18; A61K 35/16; A61K 35/19; A61K 47/34; A61P 15/08

(52) U.S. Cl. CPC A61K 38/1866; A61K 35/16; A61K 35/19; A61K 38/1808; A61K 38/1825; A61P 15/08; A61K 38/1858; A61K 38/1841; A61K 38/185; A61K 38/1833; A61K 47/34; A61K 38/18

(57) ABSTRACT

The present disclosure generally relates to the field of infertility, and in particular male infertility. Accordingly, the present disclosure provides for compositions and methods for managing male infertility, caused by azoospermia. More particularly, the present disclosure provides a therapeutic composition comprising a platelet rich plasma (PRP) or a growth factor concentrate derived therefrom and a thermoresponsive polymer. The present disclosure also relates to the compositions of PRP and the concentrate themselves. Consequently, methods to obtain the said compositions, along with therapeutic applications for treatment of azoospermia are also provided.

### TECHNICAL FIELD

The present disclosure generally relates to the field of infertility, and in particular male infertility. Accordingly, the present disclosure provides for compositions and methods for managing male infertility, caused by azoospermia. More particularly, the present disclosure provides a therapeutic composition comprising a platelet rich plasma (PRP) or a growth factor concentrate derived therefrom and a thermoresponsive polymer. The present disclosure also relates to the compositions of PRP and the concentrate themselves. Consequently, methods to obtain the said compositions, along with therapeutic applications for treatment of azoospermia are also provided.

### BACKGROUND OF THE DISCLOSURE

Inability to conceive after one year of unprotected intercourse is prevalent in approximately 15% of couples. In about 20% of infertile couples the male factor is merely responsible and in 30-40% of infertile couples it is a contributory factor. One of the conditions that inflicts infertility in males is azoospermia. Azoospermia is a medical condition characterized by absence of sperm in semen analysis. In humans, azoospermia affects about 1% of the male population and may be seen in up to 20% of male infertility conditions. Azoospermia can be classified into three major types: Pretesticular, Testicular and Post-testicular.

Pretesticular: It is characterized by inadequate stimulation of otherwise normal testicles and genital tract. Typically, follicle-stimulating hormone (FSH) levels are low (hypogonadotropic) and commensurate with inadequate stimulation of the testes to produce sperm. Pretesticular azoospermia is a kind of non-obstructive azoospermia (NOA).

Testicular: In this condition, also a type of NOA, the testes are abnormal, atrophic, or absent, and sperm production severely disturbed to absent. FSH levels tend to be elevated (hypergonadotropic) as the feedback loop is interrupted (lack of feedback inhibition on FSH). While pre- and post-testicular azoospermia are frequently correctible, testicular azoospermia is usually permanent.

Post-testicular: In this condition, sperms are produced but not ejaculated. The main cause is a physical obstruction (obstructive azoospermia) of the post-testicular genital tracts. The most common reason is a vasectomy done to induce contraceptive sterility.

Non-obstructive azoospermia (NOA) is therefore defined as a condition characterized by absence of sperm in the ejaculate due to failure of spermatogenesis and is the most severe form of male infertility. The etiology of NOA is either intrinsic testicular impairment or inadequate gonadotropin production.

NOA is generally considered a non-medically manageable cause of male infertility. Accordingly, upon evaluation, an elevated follicle-stimulating hormone (FSM level or an absence of normal spermatogenesis by testicular histology in the presence of azoospermia is generally considered sufficient evidence of a NOA.

From fertility perspective, most male patients with NOA have no therapeutic options outside of assisted reproductive techniques to conceive a biological child. Prior to microsurgical testicular sperm retrieval techniques and IVF/ICSI, donor insemination was the only option available to men with NOA. The establishment of in vitro fertilization using intracytoplasmic sperm injection (ICSI) as a standard treatment modality has resulted in a number of these men successfully fathering a child through surgically retrieved sperm from the testis. The challenge, however, is to improve their sperm count and spermatogenic function to enable the appearance of sperm in their ejaculate or to improve the chances of a successful retrieval from the testis for ICSI. Further, while options such as gene therapy, hormone treatment, surgery may be explored for production of sperm by azoospermic men, the outcome of azoospermia treatment is usually unsatisfactory. Other options that are applied by direct administration of drug and cell-based therapies also do not provide desired treatment as the said therapies are not very well explored for azoospermia and suffer from inadequate administration or retention at the site of administration. The direct injection route in these therapies mean that the drug or cells need to be in liquid form, which upon administration either leak out or are diluted by other bodily fluids.

For example, during or after intra-testes injection, the solution containing drugs and/or cells can leak out of the testis thereby decreasing the efficacy of the treatment. Moreover, growth factors and other regenerative proteins secreted by cells are released at once or over a relatively short duration of time, thereby providing a shorter duration of action. Sustained release of drugs/cells is very crucial in tissue regeneration and intended therapeutic outcome.

Regenerative medicine (RM) is offering solutions and hope for people who have conditions that today are beyond repair. RM is a game-changing area of medicine with the potential to fully heal damaged tissues and organs, with the help of stem cells and growth factors alone or together for induction of regeneration. Semen consists of spermatozoa suspended in a fluid medium called seminal plasma. Seminal plasma is a complex fluid that mediates the chemical function of the ejaculate. One component of seminal plasma is growth factors which are polypeptides functioning as paracrine, autocrine, and/or endocrine regulators of cell growth and differentiation.

IGF-1 (insulin-like growth factor-1) which has been suggested to have a direct or indirect role in spermatogenesis/steroidogenesis in the testes is one of these growth factors, so its derangement may be involved in male infertility. Also it is known that HGF (hepatocyte growth factor) and its receptor are expressed in the mammalian male genital tract. In the genital tracts of mice expression of HGF is in a region-specific manner, with slight or no expression in testes and caput epididymis, and a strong expression in corpus and cauda epididymidis. Moreover, in multiple tissues FGF (fibroblast growth factor) and FGFR (FGF receptor) expression has been reported and both, ligands and receptors role in cell proliferation, differentiation, adhesion, survival, apoptosis, and motility have been implicated. It is shown that percentage of both progressive and total motility of sperm will increase in exposure to FGF2. This effect was mediated by FGFR activation since sperm preincubation with the inhibitor prevented such increase.

Based on the ability of adult mesenchymal stem cells (MSCs) in self-renewal and multilineage differentiation, they are regarded as great candidates in the field of regenerative medicine. These cells are mainly obtained from three sources: peripheral blood, bone marrow (BM) and adipose tissue (AT). Due to invasive procedure associated complications and logistics issues, mobilised peripheral blood stein cells are used for deriving stein cell therapy protocols.

Tissue engineering traditionally stimulates cells using a single bioactive agent with key regenerative functions. For example use of G-CSF for endometrial regeneration. In contrast, natural tissue regeneration relies on a cocktail of signalling molecules and growth factors. During natural wound healing, activated platelets concentrate in the wound area and secrete a plethora of factors that play an instrumental role in not only coordinating wound healing but also in establishing normal tissue architecture and efficient tissue remodelling.

Platelet rich plasma is another option used in multiple specialities for promoting tissue regeneration.

Using a single growth factor to steer tissue regeneration represents an oversimplified and inefficient stimulus. This is generally overcome by providing supraphysiological quantities of the growth factors. As against other specialities, in ART/IVF procedures, every event is time bound and to avoid cycle cancellation, preparation of endometrium in the current cycle is very crucial which is difficult by single bioactive agent like G-CSF.

Other options that are applied by direct administration of drug and cell-based therapies also do not provide desired treatment as the said therapies are not very well explored for infertility and suffer from inadequate administration or retention at the site of administration. The direct injection route in these therapies mean that the drug or cells need to be in liquid form, which upon administration either leak out or are diluted by other bodily fluids.

Thus there is a need to combine all the effective modes of tissue regeneration with an effective delivery mechanism will benefit men suffering from poor semen quality.

For example, during or after intra-testes injection, the solution containing drugs and/or cells can leak out of the testis thereby decreasing the efficacy of the treatment. Moreover, growth factors and other regenerative proteins secreted by cells are released at once or over a relatively short duration of time, thereby providing a shorter duration of action. Sustained release of drugs/cells is very crucial in tissue regeneration and intended therapeutic outcome.

Accordingly, there is a need in the art to develop a more viable option for improving semen quality in men, which provides for better and more predictable results. Therapy that can improve quality and quantity of sperms in a semen ejaculate is highly desired and continues to be a pain point for the patient and clinicians. Since some of the commonly employed current technologies are not consistent in terms of the desired outcome, infertile men deserve a therapy that is technically advanced and can improve the current situation. Further, since drug or cell based therapies suffer from the problem of leakage, dilution and non-retention at the site of administration, a two pronged solution that achieves improvement in infertility caused by poor semen quality, by enhancing the effect of the therapy at the site of the administration may provide for a desired option.

### SUMMARY OF THE DISCLOSURE

In some embodiments, the present disclosure relates to a therapeutic composition comprising a platelet rich plasma (PRP) or a growth factor concentrate derived therefrom and a thermoresponsive polymer.

In some embodiments, the present disclosure relates to a method for preparing the therapeutic composition as recited above, comprising mixing the PRP or the growth factor concentrate derived therefrom with the thermoresponsive polymer to obtain the composition.

In some embodiments, the present disclosure relates to use of a thermoresponsive polymer for preparing a medicament for improving fertility.

In some embodiments, the present disclosure relates to a therapeutic composition comprising a platelet rich plasma (PRP) or a growth factor concentrate derived therefrom and a thermoresponsive polymer, for use in treating azoospermia in a subject in need thereof.

In some embodiments, the present disclosure relates to a method for treating azoospermia in a subject in need thereof comprising, administering to the subject the therapeutic composition of the present disclosure.

In some embodiments, the present disclosure relates to a kit for preparing the therapeutic compositions herein, comprising:
a. G-CSF;
b. a RBC activating agent selected from a group comprising: heparin, collagen, a calcium salt, hyaluronic acid, polygeline, thrombin, gelatin, EDTA, sodium citrate, starch, and a combination thereof;
c. a thermoresponsive polymer; and
d. an instruction manual.

In some embodiments, the present disclosure relates to a platelet rich plasma (PRP), wherein:
a. the PRP comprises a platelet count that is about 10 to 20-fold greater than starting whole blood sample from same subject, or
b. a red blood cell (RBC) count that is about 60 to 90-fold lower than starting whole blood sample from same subject, or
c. a white blood cell (WBC) count that is about 10 to 90-fold lower than starting whole blood sample from same subject.

In some embodiments, the present disclosure relates to a platelet-derived growth factor concentrate obtained from the PRP as recited above.

### DESCRIPTION OF THE ACCOMPANYING FIGURES

FIG. 1 represents chemical formula (A) and representation of volume phase transition (B) between coil (left) and globular (right) hydrogel conformations of a NIPAM based polymer.

FIG. 2 represents (A) the swollen PNIPAAm hydro-sol in aqueous solution below critical temperature (T_{c}) of 32° C. and (B) the shrunken dehydrated PNIPAAm hydrogel above critical temperature (T_{c}) of 32° C.

FIG. 3 represents schematic scheme for preparing the composition of the present disclosure and the subsequent administration into testis.

FIG. 4 represents impact of RBC aggregators in the PRP/GFC protocol.

FIG. 5 represents the growth factor profile of GFC.

FIG. 6 represents the in vitro growth factor release kinetics for comparing the composition of the present disclosure with a preparation devoid of the thermoresponsive polymer.

FIG. 7 represents the formation of liquid supernatant (GFC) from the PRP upon contact with platelet activating treatment.

FIG. 8 represents pictures of different stages during the protocol for preparing the PRP and GFC of the present disclosure. FIG. 8, panels A-H, show the images of various stages of whole blood processing for preparing the PRP and the GFC of the present disclosure. Panel A shows whole blood drawn from a patient and collected into into acid citrate dextrose (ACD-A) solution gel tube/K2 EDTA tube. Panel B shows settling of RBCs upon incubation of the whole blood for 45 minutes with a buffer comprising one or more RBC aggregating agents. Panel C shows the whole blood after first centrifugation at 600 rpm for 2 minutes the bottom layer contains RBCs and WBCs and the supernatant contains platelets-containing plasma. Panel D shows the supernatant containing platelets-containing plasma transferred to another centrifugation tube. Panel E shows the platelet pellet obtained after the second centrifugation step at 3000 rpm for 10 minutes. Panel F shows the gel-like consistency of PRP during the platelet-activation stage. Panels G and H show separation of platelets in the form of a clot-like structure from the supernatant containing the growth factor concentrate.

FIG. 9 represents effect of RBC aggregating agents in the protocol of the present disclosure.

### DETAILED DESCRIPTION OF THE DISCLOSURE

In view of the drawbacks associated, and to remedy the need created by the art available in the field of male infertility, the present disclosure provides compositions comprising a platelet rich plasma (PRP) and a polymer for treatment of azoospermia. In particular, the present disclosure provides compositions of the platelet rich plasma (PRP) comprising a stimulus responsive polymer.

While the PRP employed in the compositions of the present invention could be conventional, or specifically prepared as per the protocol provided in the present disclosure, the compositions can also be prepared by using a growth factor concentrate derived from the said PRP, also known as platelet-derived growth factor concentrate.

Accordingly, the present disclosure also provides compositions comprising the PRP derived growth factor concentrate along with a stimulus responsive polymer. Since the growth factor concentrate is derived from the PRP, like the PRP compositions, said compositions having the growth factor concentrate and a stimulus responsive polymer also treat azoospermia.

While the platelet rich plasma (PRP) or a growth factor concentrate derived therefrom provide for enhanced treatment of azoospermia in men by themselves, the inclusion of a stimulus responsive polymer, particularly a thermoresponsive polymer, helps in greater retention of the composition at the site of the administration. Thus, the present disclosure provides for technically advanced compositions that helps men suffering from azoospermia produce sperms at levels much higher than those observed with other currently known technologies, including use of conventional PRP without such a thermosensitive polymer.

However, before describing the compositions of the present disclosure, the corresponding methods and the applications thereof in greater detail, it is important to take note of the common terms and phrases that are employed throughout the instant disclosure for better understanding of the technology provided herein.

Throughout the present disclosure, the term “platelet rich plasma (PRP)” is used to mean conventional PRP or the PRP prepared specifically by the method of the present disclosure. Thus, unless otherwise specifically stated, the general use of the term “platelet rich plasma” or “PRP” throughout the disclosure is understood to interchangeably mean conventional PRP or the PRP prepared by the method of the present disclosure. The PRP prepared by the method of the present disclosure is also referred to herein as the “PRP prepared by the present disclosure” or the “PRP of the present disclosure”. While the method specifically employed to prepare PRP in the present disclosure will be explained in greater detail below, the conventional PRP is any PRP known in the art prepared by previously known methods and technologies, including the buffy coat method. A person skilled in the art is therefore able to refer to the literature and common general knowledge to prepare the conventional PRP quite easily. An example of methods for preparing the conventional PRP is summarized in a review article entitled “Principles and Methods of Preparation of Platelet-Rich Plasma: A Review and Author's Perspective”, J Cutan Aesthet Surg. 2014 October-December; 7(4): 189-197.

Throughout the present disclosure, the terms “growth factor concentrate” or “platelet-derived growth factor concentrate” or “platelet growth factor concentrate” or “GFC” are used interchangeably and refer to a substantially cell-free supernatant comprising a milieu of growth factors, cytokines, and other proteins obtained from lysis of activated platelets from the platelet rich plasma (PRP). As mentioned above, this PRP could be either a conventional PRP or PRP prepared by the present disclosure. The growth factor concentrate of the present disclosure is substantially free of cells as upon obtaining of the PRP, the activated platelets are lysed for the said preparation of the growth factor concentrate. The ruptured platelets are then allowed to settle down, and the substantially cell free supernatant is collected. Preferably, the growth factor concentrate is prepared from the PRP prepared by the present disclosure, which is characterized by high platelet count and very low RBC and WBC count compared to the conventional PRP. As the PRP of the present disclosure has high platelet count and very low levels of RBC and WBC contamination compared to conventional PRP the growth factor concentrate prepared from the PRP prepared by the present disclosure also has improved characteristics than growth factor concentrates prepared from conventional PRP.

Throughout the present disclosure, the term “stimulus responsive polymer” is used to mean a polymer that is sensitive to or responds to one or more stimuli, which include thermal stimuli, optical stimuli, mechanical stimuli, pH stimuli, chemical stimuli, environmental stimuli or biological stimuli. Preferably, the stimulus responsive polymers employed in the present disclosure are polymers that are sensitive or responsive to thermal stimuli. Accordingly, the stimulus responsive polymer is preferably used to mean a thermoresponsive polymer in the context of the present disclosure. These polymers are temperature-responsive polymers that exhibit a drastic and discontinuous change of their physical properties with change in temperature. For example, these polymers could be in liquid form at certain temperatures, and have the ability of quickly converting into a gel form at increased temperatures.

Throughout the present disclosure, since each of the compositions provide for a therapeutic effect in treatment of male infertility caused due to azoospermia, the term “composition” is also meant to be understood as “therapeutic composition” and the two are used interchangeably herein.

Accordingly, to reiterate, the present disclosure relates to compositions having a PRP or a growth factor concentrate derived therefrom along with a stimulus responsive polymer, preferably a thermoresponsive polymer. The said compositions are used for treatment of men suffering from infertility, caused due to azoospermia. As mentioned, the PRP employed in the compositions of the present disclosure could be a conventional PRP or a PRP prepared by the present disclosure. Accordingly, the growth factor concentrates employed in the compositions herein, are also in turn obtained from the corresponding PRP.

While the compositions of the present disclosure could comprise of conventional PRP as well as PRP prepared by the present disclosure, in some embodiments, the PRP is preferably the PRP prepared by the present disclosure. The PRP prepared by the present disclosure is enriched in platelets and comprises very low count of red blood cells (RBCS) and white blood cells (WBCs) compared to PRPs known in the art (conventional PRPs). In some embodiments, the PRP of the present disclosure comprises about 10 to 20-fold higher platelet count, 60 to 90-fold lower RBC count, and/or 10 to 90-fold lower WBC count, including values and ranges therebetween, compared to the starting whole blood sample obtained from the same subject.

In some embodiments, the PRP of the present disclosure comprises about 10, 11, 12, 13, 14, 15, 16, 17, 18, 19 or 20-fold more platelets, including values and ranges therebetween, compared to the starting whole blood sample from which the PRP is prepared. In some embodiments, the PRP of the present disclosure comprises about 10 to 12-fold, 10 to 13-fold, 11 to 14-fold, 12 to 14-fold, 12 to 15-fold and so on, more platelets, including values and ranges therebetween, compared to the starting whole blood sample. In an exemplary embodiment, if the starting whole blood sample of a subject comprises about 150×10³ platelets per microliter, the PRP prepared according to the present disclosure can comprise about 2040 platelets per microliter, which is about 13.6-fold greater than the starting whole blood sample. In another exemplary embodiment, for a whole blood sample of a subject comprising about 230×10³ platelets per microliter, the PRP of the present disclosure comprises platelets in the range of about 2300 to 3450×10³ per microliter, which is about 10 to 20-fold greater than the starting whole blood sample.

The PRP of the present disclosure is preferably autologous. However, allogenic PRP and use of allogenic PRP is also contemplated. In some embodiments, the PRP is prepared from venous blood. In some embodiments, the PRP is prepared from cord blood or bone marrow. In some embodiments, the PRP is derived from umbilical cord blood, bone marrow or fresh or expired platelet concentrates from blood banks.

It is known in the art that platelets serve as a reservoir of growth factors, cytokines, and other proteins. These growth factors, cytokines, and several other proteins are contained in the alpha-granules of platelets and are released upon activation of platelets. Exemplary growth factors present in the growth factor concentrate of the present disclosure include, but are not limited to, platelet-derived growth factor (PDGF), transforming growth factor (TGF), platelet-derived angiogenesis factor (PDAF), vascular endothelial growth factor (VEGF), epidermal growth factor (EGF), insulin-like growth factor (IGF), basic fibroblast growth factor (bFGF), stromal cell derived factor 1 (SDF-1), and hepatocyte growth factor (HGF). Accordingly, in some embodiments, the compositions herein comprise the growth factor concentrate obtained from PRP along with the thermoresponsive polymer.

The present disclosure therefore provides a therapeutic composition having the GFC the thermoresponsive polymer, wherein the growth factor concentrate comprises growth factor(s) selected from a group comprising VEGF, EGF, bFGF, IGF-1, PDGF-BB and TGF-b1 or any combination thereof.

As mentioned, the GFC employed in the present disclosure is prepared from the PRP, which could be conventional PRP or the PRP prepared by the present disclosure. The GFC is prepared by subjecting the activated platelets in the PRP to one or more platelet-activating treatments. These are described in further details in the later paragraphs of the present disclosure.

As the term suggests, the GFC is a concentrated form of growth factors that are originally present in the platelets. Upon platelet-activating treatment, the activated platelets release the said growth factors in the plasma. Accordingly, the concentration of the growth factors in the GFC is about 4 to 10-fold, about 4 to 8-fold, about 5 to 10-fold, about 5 to 8-fold, about 6 to 10-fold, or about 6 to 8-fold, including values and ranges therebetween, higher than that of the starting whole blood sample.

As was the case with PRP, while the GFC can be prepared from conventional PRP, in some embodiments, it is preferred that the GFC is obtained from the PRP prepared by the present disclosure. Exemplary levels of certain growth factors in the growth factor concentrate of present disclosure are shown in the table 1 below:

TABLE 1

| | Concentration range in the | | | |
|---|---|---|---|---|
| Growth | freshly-prepared GFC derived | | Concentration range in the | |
| Factor | from conventional PRP | | freshly-prepared GFC | |
| VEGF | 500-800 | pg/mL | 500-1300 | pg/mL |
| EGF | 100-200 | pg/mL | 100-2000 | pg/mL |
| bFGF | 25-75 | pg/mL | 25-500 | pg/mL |
| IGF-1 | 70-130 | ng/mL | 500-1000 | ng/mL |
| PDGF-BB | 20-85 | ng/mL | 20-500 | ng/mL |
| TGF-β1 | 250-350 | ng/mL | 100-2000 | ng/mL |

Thus, in the therapeutic composition of the present disclosure comprising the GFC and the thermoresponsive polymer, concentration of the VEGF ranges from about 500-1300 pg/mL, concentration of the EGF ranges from about 100-2000 pg/mL, concentration of the bFGF ranges from about 25-500 pg/mL, concentration of the IGF-1 ranges from about 500-1000 ng/mL, concentration of the PDGF-BB ranges from about 20-500 ng/mL, and concentration of the TGF-b1 ranges from about 100-2000 ng/mL. Apart from the PRP or the GFC and the thermoresponsive polymer, the compositions of the present disclosure also comprise peripheral blood stem cells (PBSCs) or endothelial progenitor cells. These PBSCs are a direct result of Endogenous Stem Cell Mobilisation (ESCM) done prior to preparing of the composition. Combining the compositions with PBSCs proves to be more effective as it ensures local availability of the composition for a longer time thereby ensuring improved sperm maturation and vitality. Accordingly, the therapeutic compositions of the present disclosure comprise of PBSCs in addition to the PRP or the growth factor concentrate, along with the thermoresponsive polymer.

In some embodiments, concentration of the PBSCs or the endothelial progenitor cells within the therapeutic composition of the present disclosure ranges from about 10% to 50%. It is important to note that the compositions of the present disclosure comprise of PRP or GFC, which are derived from whole blood of a subject. Accordingly, as is well known and understood by a person skilled in the art, the internal composition of the whole blood, including the number of cells, proteins, active agents, growth factors etc. varies from subject to subject. Therefore, the PRP or the GFC so prepared varies accordingly, and so do the additional elements, including the PBSCs, and thus arises a need for a range of concentrations within which the compositions of the present disclosure can be prepared and applied. Accordingly, within the ambit of the present disclosure, the concentration of the PBSCs can be any of 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%.

In the context of the present disclosure, the percentage concentration of the PBSCs recited herein in intended to convey the final composition of the PBSC containing solution as part of the compositions of the present disclosure. In other words, for example if the concentration of the PBSCs or the endothelial progenitor cells within the therapeutic composition of the present disclosure is at about 30%, it means that about 30% of the final therapeutic composition is made up of the solution containing the PBSCs. As a person skilled in the art understands the importance of PBSCs and the number of cells in the whole blood versus the solution of PBSCs so prepared, the final number of cells per se provided in the therapeutic composition can be calculated based on the percentage of the solution accordingly. Similarly, when the solution of PBSCs is prepared by the buffy coat method as described in the present disclosure, the level of WBCs in the PBSCs increase multi-folds when compared to the corresponding whole blood levels. Therefore, the final number of cells per se provided in the therapeutic composition can be calculated based on the percentage of the solution accordingly.

In order for the PBSCs or the endothelial progenitor cells to be included in the compositions of the present disclosure, Bone-Marrow Derived Stem Cell (BMSC) mobilization is stimulated by Granulocyte-Colony Stimulating Factor (G-CSF). These cells migrate into affected tissues and contribute to tissue repair. Accordingly, in a non-limiting embodiment, prior to the withdrawal of blood for preparation of the compositions of the present disclosure, the subject is administered with Granulocyte-Colony Stimulating Factor (G-CSF).

G-CSF is a cytokine secreted by various tissues that stimulates the proliferation, differentiation and function of neutrophil precursors and mature neutrophils. G-CSF naturally stimulates BMSC mobilization. Contrary to most tissues in which SDF-1 is secreted consequent to an injury or a degenerative condition, in the bone marrow SIF-1 is constitutively produced and released, and binding of SDF-1 to its exclusive receptor CXCR4 leads to the externalization of adhesion molecules, namely integrins, which allow for the adherence of stem cells to the bone marrow matrix. The binding of SDF-1 to CXCR4 is referred to as the SDF-1/CXCR4 axis. The general understanding is that disruption of the SDF-1/CXCR4 axis reduces the expression of adhesion molecules, leading to a reduction in the adherence of stem cells to the bone marrow matrix and the consequent mobilization of stem cells.

Various compounds known to trigger stem cell mobilization all affect the SDF-1/CXCR4 axis in various ways. For example, G-CSF disrupts the SDF-1/CXCR4 axis by activating a series of proteolytic enzymes including elastase, cathepsin G, and various matrix metalloproteinases (MMP2 and MMP9) that inactivate SDF-1 (Marinello et al., 2006; Tin et al., 2006; Cation et al., 2003).

In some embodiments, administration of G-CSF enhances the concentration of WBCs in the blood by about 5-folds, when compared to whole blood analysed without stimulation by G-CSF.

Without wishing to be bound by any theory, it is understood that the naturally mobilized BMSC can traffic to various areas of the body and contribute to tissue regeneration and repair through peripheral blood as peripheral blood stem cells (PBSCs). As PBSCs play a key role in the process of SC-mediated tissue repair, employing PBSCs in a tissue regenerative composition like the ones of the present disclosure constitutes a therapeutic approach. In view of said rationale, in an embodiment of the present disclosure, a portion of the withdrawn blood is employed to isolate PBSCs; which are then included as part of the compositions of the present disclosure.

In some embodiments, the PBSCs employed in the present disclosure are autologous, or are derived from umbilical cord blood, bone marrow, or huffy coat from blood banks.

In exemplary embodiments, said isolated PBSCs are added to the platelet derived growth factor concentrate for therapeutic applications. The aspect of isolation of PBSCs and their combination with the platelet derived growth factor concentrate of the present disclosure is performed by methods generally known in the art. This is further elaborated on in further sections of the present disclosure.

Thus, the present disclosure provides compositions that comprise thermosenstive polymer; conventional PRP or PRP prepared by the present disclosure or the GFC obtained from either of the two PRPs; and peripheral blood stem cells (PBSCs).

In some embodiments of the present disclosure, the compositions herein also comprise one or more additional therapeutic agent selected from a group comprising hormone, growth factor, protein, cell, cell secretome, and drug, or any combination thereof. For example, the composition can comprise any one or more of follicle stimulating hormone (FSH), luteinizing hormone (LH), high-density lipoprotein (HDL), steroidogenic acute regulatory protein (StAR), stem cell, phosphodiesterase V inhibitors, sildenafil citrate, 1 adrenergic blocker and alprostadil. The stem cells may include adult or embryonic stem cells and from varied sources including those from bone marrow, adipose tissue, blood, umbilical cord and embryo. Further, any drug that is a therapeutic agent known to a person skilled in the art for the treatment of azoospermia, and which can be employed without any compatibility challenges with the compositions of the present disclosure, are also contemplated within the ambit of the present disclosure.

In some embodiments, the growth factors that are included as additional therapeutic agents in the compositions of the present disclosure include Vascular Endothelial Growth Factor (VEGF), Nerve Growth Factor (NGF), Fibroblast Growth Factor (FGF), Hepatocyte Growth Factor (HGF), Insulin-like growth factor (I-IGF-1), Epithelial Growth Factor (EGO, Platelet Derived Growth. Factor (PDGF), Transforming growth factor-b/family and Stem cell growth factor (SGF). In some embodiments, these growth factors help in fortification of the final composition and help in enhancing the overall concentration of the growth factors in the final preparation. As the compositions of present disclosure comprise of PRP or GFC, which are derived from whole blood of a subject, it is well known and understood by a person skilled in the art that the internal composition of the whole blood, including the number of growth factors vary from subject to subject. Accordingly, the growth factors as part of the additional therapeutic agents help in maintaining the overall levels of growth factors in the final composition.

In some embodiments, when the additional therapeutic agent is a hormone, protein, cell, cell secretome, or drug, or any combination thereof, they are present in the composition at a concentration ranging from about 10% to 50% of the composition. Accordingly, within the ambit of the present disclosure, the concentration of the additional therapeutic agent in the composition can be any of 20%, 21%, 22%, 23%, 24%, 25%, 26%, 27%, 28%, 29% or 30%. However, when the additional therapeutic agent is a growth factor, it is present at a concentration which is about 4-fold to 10-fold higher than the physiological levels of the constituting whole blood used to prepare the compositions. According, within the ambit of the present disclosure, the concentration of the growth factor in the composition can be any of 4-fold, 5-fold, 6-fold, 7-fold, 8-fold, 9-fold or 10-fold.

In some embodiments, in addition to growth factors from autologous blood, therapeutic compositions are further fortified with exogenously added growth factors to provide a concentration of growth factors that is about 4 to 10 times higher than the baseline concentration of corresponding growth factors in starting whole blood. Accordingly, in some embodiments, in the therapeutic compositions, concentration of the VEGF ranges from about 500 to 3000 pg/mL, concentration of the EGF ranges from about 100 to 3000 pg/mL, concentration of the bFGF ranges from about 25 to 3000 pg/mL, concentration of the IGF-1 ranges from about 500 to 3000 ng/mL, concentration of the PDGF-BB ranges from about 20 to 3000 ng/mL, and concentration of the TGF-β1 ranges from about 100 to 3000 ng/mL.

Thus, the present disclosure provides compositions that comprise thermosenstive polymer; conventional PRP or PRP prepared by the present disclosure or the GFC obtained from either of the two PRPs; peripheral blood stem cells (PBSCs), and one or more additional therapeutic agent. While the present disclosure provides for compositions as captured in this or the previous embodiments, it is important to note that the present disclosure equally contemplates all other possible permutations-combinations that may be possible from the present disclosure, as long as the compositions at minimum comprise conventional PRP or PRP prepared by the present disclosure or GFC obtained from either of the two PRPs; and thermosenstive polymer. Thus, all compositions that comprise both peripheral blood stem cells (PBSCs), and one or more additional therapeutic agent, or comprise only PBSCs without any additional therapeutic agent or comprise only one or more additional therapeutic without any PBSCs are within the ambit of the present disclosure.

Accordingly, while PRP or GFC forms the active center of the compositions, it is the thermosensitive polymer that enhances the therapeutic effect by ensuring that the composition is retained by the body at the site of administration for a longer period of time. Since the polymer is thermosensitive in nature, one of the most important properties that it showcases is the conversion of its physical form from liquid to gel, when in contact with physiological temperature. Thus, in some embodiments, while it is viscous but in the form of an injectable liquid at room temperature, it transitions to a temporary self-forming polymeric plug at body temperature. For example, the thermoresponsive polymer exists in a liquid form at a temperature ranging from about −20° C. to +27° C., and in a gel form at a temperature ranging from about +27.1° C. to +60° C. Because the material undergoes a temperature-induced phase change with no alteration in the product's chemical composition, it works well to enhance the overall impact of the composition. The use of thermoresponsive polymers in the present disclosure therefore allows for sustained and targeted effect of the therapeutic composition of the present disclosure and prevents leakage from the site of administration or dilution by other bodily fluids.

In some embodiments, the thermoresponsive polymer employed to prepare the compositions of the present disclosure is a synthesized biocompatible polymer, which have no biological contaminants. An example of such a polymer is N-isopropylacrylamide (NIPAM) based polymer, for instance poly(Nisopropylacrylamide-co-n-butyl methacrylate) poly(NIPAAm-co-BMA). The present disclosure therefore provides for compositions that comprise a NIPAM based polymer; conventional PRP or PRP prepared by the present disclosure or the GFC obtained from either of the two PRPs; optionally along with peripheral blood stem cells (PBSCs), and one or more additional therapeutic agent.

In some embodiments, a thermoresponsive polymer employed to prepare the compositions of the present disclosure includes copolymers composed of thermoresponsive polymer blocks and hydrophilic polymer blocks and is characterized by its temperature-dependent dynamic viscoelastic properties. The thermoresponsive polymer blocks are hydrophilic at temperatures below the sol-gel transition temperature and are hydrophobic at temperatures above the sol-gel transition temperature. The hydrophobic interaction results in formation of a homogenous three-dimensional polymer network in water. In some embodiments, the thermoresponsive polymer block which are part of such copolymers is a NIPAM based polymer. An example of such thermoresponsive polymer blocks is poly(Nisopropylacrylamide-co-n-butyl methacrylate) poly(NIPAAm-co-BMA), which are combined with hydrophilic polymer blocks, including polyethylene glycol (PEG). The present disclosure therefore provides for compositions that comprise a copolymer of poly(Nisopropylacrylamide-co-n-butyl methacrylate) poly(NIPAAm-co-BMA) and polyethylene glycol (PEG); conventional PRP or PRP prepared by the present disclosure or the GFC obtained from either of the two PRPs; optionally along with peripheral blood stem cells (PBSCs), and one or more additional therapeutic agent. As alternatives to PEG, the thermoresponsive polymers can also comprise poly(D,L-lactide-co-glycolide) (PLGA), polylactic acid) (PLA), poly(glutamic acid) (PGA), poly(caprolactone) (PCL), N-(2-hydroxypropyl)-methacrylate (HPMA) copolymers, and poly(amino acids). In some embodiments, PEGylated NIPAM polymers can be prepared as described by the methods known in the art.

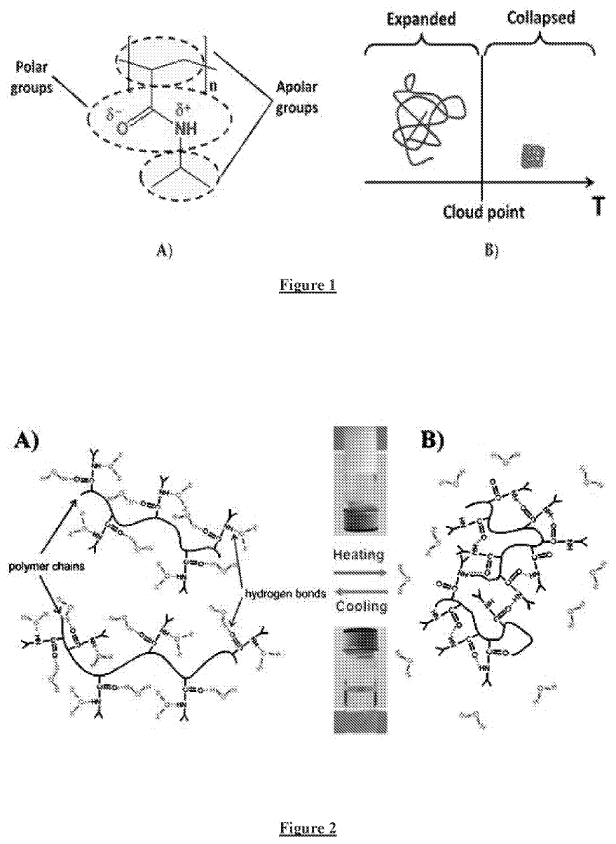

In some embodiments, chemical formula (A) and representation of volume phase transition (B) between coil (left) and globular (right) hydrogel conformations of a NIPAM based polymer is provided by FIG. 1. Further, representation of (A) the swollen PNIPAAm hydro-sol in aqueous solution below critical temperature (T_{c}) of 32° C. and (B) the shrunken dehydrated PNIPAAm hydrogel above critical temperature (T_{c}) of 32° C. is provided by FIG. 2.

In some embodiments, the thermoresponsive polymer employed to prepare the compositions of the present disclosure include amphiphilic block copolymers, or ABA triblock copolymers including poloxamers, such as poloxamer 407. These polymers are biocompatible, highly water-soluble and polymorphic materials, and thus ideal for us in thermo sensitive biological applications. While they dissolve conveniently in blood, they are also excreted easily m Urine.

In some embodiments, the amphiphilic copolymers include those with hydrophilic block hydrophobic block polymers. An example of such an amphiphilic polymer is a copolymer of poly(ethylene oxide) (PEO) and polypropylene oxide) (PPO). A commercially available example of such a polymer is Plutonic®.

In some embodiments, a thermoresponsive polymer employed to prepare the compositions of the present disclosure includes any polymer known to a person skilled in the art that possesses thermoresponsive properties. The present disclosure accordingly also contemplates all thermoresponsive polymers that are known in the art, commercially available and/or those approved for medical/therapeutic applications by the U.S. Food and Drug Administration (FDA).

While the presence of the thermoresponsive copolymer is a mandatory feature of the compositions of the present disclosure, the concentration at which the polymer may be present within the composition can vary over a range depending on the final constituents of the composition, including PRP, GFC, PBSCs and/or additional therapeutic agents. Similarly, the concentration of the PRP and the GFC within the composition also varies over a specified range.

Thus, in some embodiments, concentration of the thermoresponsive polymer within the therapeutic composition of the present disclosure ranges from about 10% to 90%, whereas concentration of the PRP or the GFC within the therapeutic composition of the present disclosure ranges from about 10% to 90% Accordingly, the PRP or the GFC and the thermoresponsive polymer are present in the compositions of the present disclosure at a ratio ranging from about 90:10 to 10:90.

Since the ratio of the PRP or GFC and the thermoresponsive polymer varies from composition to composition depending on the initial constituents of PRP or GFC, and the final application, the present disclosure contemplates all such compositions that satisfy the concentration and ratio requirements set out above. It is important to note that the compositions of the present disclosure comprise of PRP or GFC, which are derived from whole blood of a subject. Accordingly, as is well known and understood by a person skilled in the art, the internal composition of the whole blood, including the number of cells, proteins, active agents, growth factors etc. varies from subject to subject. Therefore, the PRP or the GFC so prepared varies accordingly, and thus arises a need for a range of concentrations within which the compositions of the present disclosure can be prepared and applied. Accordingly, within the ambit of the present disclosure, the concentration of the thermoresponsive polymer can be any of 10%, 11%, 12%, 13%, 14%, 15%, 16%, 17%, 18%; 19%, 20%, 21%, 22%, 23%, 24%, 25%, 26%, 27%, 28%, 29%, 30%, 31%, 32%, 33%, 34%, 35%, 36%; 37%, 38%, 39%, 40%, 41%, 42%, 43%, 44%, 45%, 46%, 47%, 48%, 49% or 50%. Also, Accordingly, within the ambit of the present disclosure, the concentration of the PRP or the GFC can be any of 10%, 11%, 12%, 13%, 14%, 15%, 16%, 17%, 18%, 19%, 20%; 21%, 22%; 23%, 24%, 25%, 26%, 27%, 28%, 29%, 30%, 31%, 32%, 33%, 34%, 35%, 36%, 37%, 38%, 39%, 40%; 41%, 42%, 43%, 44%, 45%, 46%, 47%, 48%, 49% or 50%, 51%, 52%, 53%, 54%, 55%, 56%, 57%, 58%, 59%, 60%, 61%, 62%, 63%, 64%, 65%, 66%, 67%, 68%, 69%; 70%, 71%, 72%, 73%, 74%, 75%, 76%, 77%, 78%, 79%, 80%, 81%, 82%, 83%, 84%, 85%, 86%, 87%, 88%, 89% or 90%.

The present disclosure therefore provides for compositions that comprise a thermoresponsive polymer at a concentration ranging from about 10% to 50%; conventional PRP or PRP prepared by the present disclosure or the GFC obtained from either of the two PRPs at a concentration ranging from about 10% to 50%; optionally along with peripheral blood stem cells (PBSCs) at a concentration ranging from about 10% to 50%, and one or more additional therapeutic agents at a concentration ranging from about 20% to 30% of the composition. For example, a composition herein can comprise a thermoresponsive polymer at a concentration of about 20%; conventional PRP or PRP prepared by the present disclosure or the GFC obtained from either of the two PRPs at a concentration of about 30%; along with peripheral blood stem cells (PBSCs) or the endothelial progenitor cells at a concentration of about 50%.

Now, as mentioned previously, the therapeutic compositions of the present disclosure are helpful in treatment of male infertility, by treating azoospermia in a subject in need thereof. Accordingly, the present disclosure provide a composition comprising a platelet rich plasma (PRP) or a growth factor concentrate derived therefrom and a thermoresponsive polymer, for use in treating azoospermia in a subject in need thereof.

In some embodiments, the compositions for use in treatment of azoospermia are identical to those envisaged in the present disclosure. Accordingly, the present disclosure provides for compositions that comprise a thermoresponsive polymer at a concentration ranging from about 10% to 50%; conventional PRP or PRP prepared by the present disclosure or the GFC obtained from either of the two PRPs at a concentration ranging from about 10% to 50%; optionally along with peripheral blood stem cells (PBSCs) at a concentration ranging from about 10% to 50%, and one or more additional therapeutic agents at a concentration ranging from about 20% to 30% of the composition, for use in treatment of azoospermia.

Now in order for the composition of the present disclosure to be manufactured, the present disclosure also provides a method for preparing the therapeutic composition comprising a thermoresponsive polymer; conventional PRP or PRP prepared by the present disclosure or the GFC obtained from either of the two PRPs; optionally along with peripheral blood stem cells (PBSCs), and one or more additional therapeutic agents. The method comprises mixing the PRP or the growth factor concentrate derived therefrom with the thermoresponsive polymer, optionally along with the PBSCs and additional therapeutic agents, to obtain the composition.

In some embodiments, the mixing of the components to prepare the composition of the present disclosure is carried out by adding the PRP or GFC in a concentration ranging from about 10% to 50% directly to the thermoresponsive polymer under sterile environment. While this thermoresponsive polymer is prepared separately in a liquid selected from water or saline, such as PBS, prior to its mixing with the PRP or the GFC, it is important to note that the concentration of the thermoresponsive polymer must also remain between about 10% to 50% in the final therapeutic composition of the present disclosure.

In some embodiments, depending on the end application of the therapeutic compositions of the present disclosure, the thermoresponsive polymer employed to prepare the composition is in the form of a powder, which is subjected to mixing with water or saline, including PBS, to form a liquid. This liquid is subsequently mixed with the PRP or the GFC to obtain the composition of the present disclosure. However, in alternative embodiments, the thermoresponsive polymer may remain in the form of a powder and mixed directly with the PRP or the GFC to obtain the composition of the present disclosure. In any case, the concentrations of the thermoresponsive polymer within the compositions herein remain within the range provided in the disclosure herein.

In some embodiments, a method for preparing a polymer solution as mentioned above comprises steps of: a) combining an amount of the thermoresponsive polymer or a combination of two polymers (such as NIPAM and PEG) with an amount of a suitable aqueous solvent fortified with growth factors, wherein the amount of polymer(s) is sufficient to form a solution having up to about 10% to 50% w/w of polymer(s); b) stirring the mixture at a sufficiently medium speed at about or below 10° C. at for a first period of time; and c) rocking the mixture for a second period of time thereby forming a solution.

Post contacting of the thermoresponsive polymer with the PRP or GFC, the mixture is cooled in refrigerator or over ice at a temperature ranging from about 2° C. to 10° C. for about 15 minutes. The tube is periodically shaken to help mixing of the contents. Upon dissolving, the mixture is allowed to settle for elimination of air bubbles, post which the mixture, or the composition, is ready for therapeutic administration. As mentioned, once the thermoresponsive polymer is prepared in the solution form or is obtained in the powder form, it is combined with the PRP or the GFC for preparing the compositions of the present disclosure. Accordingly, the present disclosure also provides for use of the thermoresponsive polymer for preparing the therapeutic composition of the present disclosure.

Thus, in some embodiments, the present disclosure provides for use of the thermoresponsive polymer for preparing a medicament for improving fertility. More particularly, the present disclosure provides for use of the thermoresponsive polymer for preparing a medicament which is the therapeutic composition of the present disclosure for improving fertility in men.

In some embodiments, the present disclosure provides for use of the thermoresponsive polymer for preparing a therapeutic compositions for treating infertility caused by azoospermia, wherein the polymer is mixed along with a platelet rich plasma (PRP) or a growth factor concentrate derived therefrom. Of course, in case the compositions of the present disclosure comprise PBSCs and/or additional therapeutic agent(s), the said components also become part of such compositions.

In some embodiments, the medicament prepared by using the thermoresponsive polymer improves fertility by treating azoospermia.

In some embodiments, a composition comprising the platelet rich plasma (PRP) or the platelet-derived growth factor concentrate (GFC) along with pharmaceutically acceptable excipients, can also be used for administration to a subject, for treatment of azoospermia.

Since the compositions herein also contemplate inclusion of PBSCs or the endothelial progenitor cells and/or one or more additional therapeutic agents, the present disclosure also provides for methods for said inclusion(s) accordingly.

In some embodiments, the PBSCs are added to the compositions of the present disclosure comprising the thermoresponsive polymer and PRP or GFC just prior to administration of the said composition to a subject suffering from azoospermia. In some embodiments of the present disclosure, once the whole blood is collected for the preparation of the PRP or the GFC, a fraction of the blood is kept aside for the preparation of endothelial progenitor cells or PBSCs.

In some embodiments, the PBSCs are added to the PRP or GFC of the present disclosure, followed by mixing with thermoresponsive polymer just prior to administration of the said composition to a subject suffering from azoospermia. In some embodiments of the present disclosure, once the whole blood is collected for the preparation of the PRP or the GFC, a fraction of the blood is kept aside for the preparation of endothelial progenitor cells or PBSCs, which is later mixed with the PRP or GFC, prior to the mixture being contacted with the polymer.

As mentioned previously, since the subject is administered G-CSF one to three days prior to the administration of the composition, the Bone-Marrow Derived Stem Cells (BMSCs) are mobilized leading to circulation of the PBSCs in the blood.

On the day of the administration, the said blood is withdrawn and subjected to conventional protocols for harvesting of the PBSCs. The said conventional protocols include those that provide for removal of PBSCs by huffy coat preparation.

Accordingly, in some embodiments, the PBSCs are prepared in a solution form by the following huffy coat protocol comprising steps of:
a) incubating whole blood collected in an anti-coagulant container with a red blood cell (RBC) aggregating agent selected from the group consisting of: heparin, collagen, a calcium salt, hyaluronic acid, polygeline, thrombin, gelatin, EDTA, sodium citrate, starch, and a combination thereof;
b) subjecting the whole blood to centrifugation at speed of about 1200 rpm for about 15 minutes;
c) allowing formation of three layers as per cell density of the blood, comprising a bottom layer consisting of RBCs, a middle layer consisting of platelets and WBCs, and a top layer comprising platelet poor plasma (PPP);
d) removing top layer containing platelet-poor plasma and transferring middle buffy-coat layer containing PBSCs to another sterile tube; and
e) subjecting the buffy coat layer to centrifugation at a speed of about 2000 rpm for about 10 minutes or filtration to separate PBSCs to obtain a solution comprising the PBSCs.

In some embodiments, the whole blood is stored or maintained at a temperature ranging from about 20° C. to 24° C. prior to and/or during the preparation the PBSCs.

Once the solution comprising the PBSCs is prepared, it is mixed with the composition of the thermoresponsive polymer and PRP or GFC at a concentration ranging from about 10% to 50%.

As the compositions herein, in some embodiments, comprise the PRP prepared by the present disclosure, in order for the said composition to be manufactured, the present disclosure also provides a method for preparing the PRP of the present disclosure. Accordingly, the present disclosure also relates to a method for preparing a PRP, wherein the PRP comprises a platelet count that is about 10 to 20-fold greater than starting whole blood sample, or a RBC count that is about 60 to 90-fold lower than starting whole blood sample, and/or a WBC count that is about 10 to 90-fold lower than starting whole blood sample. The method broadly comprises treating a whole blood sample with one or more RBC aggregating agents, spinning the blood to sediment RBCs and WBCs, spinning the supernatant to sediment platelets, and resuspending the platelets in platelet-poor plasma to provide the PRP.

In one embodiment, the method for preparing PRP comprises: (a) incubating a whole blood sample collected in an anti-coagulant container with RBC aggregating agent(s); (b) subjecting the whole blood sample incubated with the RBC aggregating agent to a first centrifugation step to obtain a supernatant containing platelets; (c) subjecting the supernatant to a second centrifugation step to obtain a platelet pellet and platelet-poor plasma (PPP); and (d) resuspending the platelet pellet in PPP to obtain the PRP.

The above described order of steps is not binding on the method of the present disclosure and does not restrict the order in which the steps must be performed. The steps may be performed in any order that is logically feasible, and known to a person skilled in the art.

In some embodiments, the RBC aggregating agent is selected from a group comprising heparin, collagen, a calcium salt, hyaluronic acid, polygeline, thrombin, gelatin, EDTA, sodium citrate, starch, and any combination thereof. In an exemplary embodiment, the RBC aggregating agent is a combination of heparin, collagen, and a calcium salt. In another exemplary embodiment, the RBC aggregating agent is a combination of hyaluronic acid, polygeline, thrombin. In another exemplary embodiment, the RBC aggregating agent is a combination of polygeline, thrombin, and gelatin. In another exemplary embodiment, the RBC aggregating agent is a combination of thrombin, gelatin, and sodium citrate. In another exemplary embodiment, the RBC aggregating agent is a combination of heparin, polygeline, and starch. In another exemplary embodiment, a RBC aggregating agent is a combination of polygeline, gelatin, and starch. In some embodiments, the RBC activating agent is suspended in a physiologically acceptable buffer. In some embodiments, the RBC activating agent is added to the whole blood at a concentration of about 0.2% to 30%, for example, about 0.2, 0.4, 0.6, 0.8, 1.0, 2, 3, 4, 5, 6, 7, 8, 9, or 10%, 20% and 30% by volume of the whole blood sample. In some embodiments, the concentration range of the stock ranges from about 10% to 100%. The whole blood sample is incubated with the RBC activating agent for about 5 to 45 minutes at an ambient temperature. The ambient temperature for incubation ranges from about 4° C. to 37° C., about 10° C. to about 20° C. about 20° C. to 30° C. or about 20° C. to about 25° C. The time of incubation ranges from 5 to 45 minutes, including values and ranges therebetween, such as about 15 minutes, about 20 minutes, about 25 minutes, about 30 minutes, about 35 minutes, about 40 minutes, about 45 minutes, about 15 to 45 minutes, about 30 to 45 minutes, about 10 to 40 minutes, or about 20 to 40 minutes. During the incubation, RBCs aggregate and start settling down.

After incubation with the RBC aggregating agent, the whole blood sample is centrifuged (first centrifugation) at a low speed such as about 300-1000 rpm for about 2-10 minutes. In some embodiments, the first centrifugation step is carried out at about 300 to 1000 rpm, about 350 to 950 rpm, about 350 to 800 rpm, about 400 to 900 rpm, about 450 to 950 rpm, about 400 to 800 rpm, about 500 to 1000 rpm, about 500 to 900 rpm, about 500 to 850 rpm, about 500 to 800 rpm, about 550 to 750 rpm, about 550 to 700 rpm, about 550 to 800 rpm, about 600 to 800 rpm, about 650 to 800 rpm, or about 650 to 750 rpm, including values and ranges therebetween.

Time for the first centrifugation step ranges from about 2 to 10 minutes, about 2 to 8 minutes, about 2 to 6 minutes, about 2 to 5 minutes, about 2 to 4 minutes, about 2 to 3 minutes, about 3 to 9 minutes, about 3 to 8 minutes, about 3 to 5 minutes, about 3 to 4 minutes, about 4 to 8 minutes, about 5 to 10 minutes, including values and ranges therebetween. The first centrifugation step can be carried out at any of the speed values for any of the time periods described herein. In the first centrifugation step, RBCs and WBCs sediment and platelets remain in the supernatant. Treatment with RBC aggregating agents prior to the first centrifugation ensures efficient removal of RBCs from the Whole Blood by way of sedimentation.

After the first centrifugation step, the supernatant containing platelets is further centrifuged (second centrifugation step) to sediment platelets. The second centrifugation step is carried out at about 1200 to 3500 rpm for about 5-15 minutes. In some embodiments, the second centrifugation step is carried out at about 1200 to 5000 rpm for about 5-15 minutes. In some embodiments, the second centrifugation step is carried out at about 1200 to 5000 rpm, 1200 to 4500 rpm, 1200 to 4000 rpm, 1200 to 3500 rpm, about 1200 to 3200 rpm, about 1400 to 3500 rpm, about 1400 to 3200 rpm, about 1500 to 3500 rpm, about 1500 to 3200 rpm, about 1500 to 3000 rpm, about 1800 to 3500 rpm, about 1800 to 3200 rpm, about 1800 to 3000 rpm, about 2000 to 3000 rpm, about 2200 to 3200 rpm, about 2500 to about 3200 rpm, about 2500 to 3000 rpm, about 2800 to 3200 rpm, about 2900 to 3100 rpm, including values and ranges therebetween for about 5 to 15 minutes, about 5 to 12 minutes, about 5 to 10 minutes, about 6 to 12 minutes, about 6 to 10 minutes, about 8 to 1.5 minutes, about 8 to 12 minutes, about 10 to 15 minutes, about 10 to 12 minutes, or about 12 to 15 minutes, including values and ranges therebetween. After the second centrifugation step, platelets form a pellet leaving platelet-poor plasma (PPP) as supernatant. PPP is aspirated and a desired volume of PPP is used to resuspend the platelet pellet to provide platelet-rich plasma. In some embodiments, platelet pellets obtained from about 30 to 60 ml of starting whole blood sample are resuspended in about 3 ml to 6 ml of PPP to provide PRP.

In some embodiments, a method for preparing PRP comprises: (a) incubating a whole blood sample collected in an anti-coagulant container with RBC aggregating agent(s) selected from a group comprising heparin, collagen, a calcium salt, hyaluronic acid, polygeline, thrombin, gelatin, EDTA, sodium citrate, starch, and any combination thereof, wherein the incubation is carried out at a temperature of about 20-25° C.; (b) subjecting the whole blood sample incubated with the RBC aggregating agent to a first centrifugation step to obtain a supernatant containing platelets, wherein the first centrifugation is carried out at about 300-1000 rpm for about 2-10 minutes; (c) subjecting the supernatant to a second centrifugation step to obtain a platelet pellet and platelet-poor plasma (PPP), wherein the second centrifugation is carried out at about 1200-3500 rpm for about 5-15 minutes; and (d) resuspending the platelet pellet in PPP to obtain the PRP. Said method for preparing the PRP described herein provides about 10 to 20-fold enrichment of platelets compared to starting whole blood sample, or about 60 to 90-fold reduction in the RBC count compared to starting whole blood sample, and/or about 10 to 90-fold reduction in WBCs, including values and ranges therebetween, compared to stalling whole blood sample from same subject.

Accordingly, the present disclosure relates to PRP so prepared by the method of e present disclosure. As mentioned, in some embodiments, the number of platelets, RBCs, and/or WBCs present in the PRP of the present disclosure are characterized in terms of fold increase or fold decrease compared to the starting whole blood sample or conventional PRPs as the number of platelets, RBCs, and WBCs vary from a subject to subject or even for the same subject over the period of time; accordingly, a fold increase/enrichment (for platelets) and/or a fold decrease/reduction. (for RBCs/WBCs) effectively characterize or distinguish the PRP of the present disclosure over starting whole blood sample and/or conventional PRPs.

In some embodiments, the platelet count of the PRP of the present disclosure is about 1.2 to 2.5-fold, including values and ranges therebetween, greater than the platelet count of the conventional PRP. In some embodiments, the platelet count of the PRP of the present disclosure is about 1.2, 1.3, 1.4, 1.5, 1.6, 1.7, 1.8, 1.9, 2, 2.1, 2.2, 2.3, 2.4, or 2.5-fold, including values and ranges therebetween, greater than the platelet count of the conventional PRP. In some embodiments, the platelet count of the PRP of the present disclosure is about 1.2 to 2.2-fold, about 1.2 to 2-fold, about 1.2 to 1.8-fold, about 1.2 to 1.6-fold, about 1.5 to 2.5-fold, 1.5 to 2.2-fold, about 1.5 to 2-fold, including values and ranges therebetween, greater than the platelet count of the conventional PRP.

In some embodiments, the RBC count of the PRP of the present disclosure is about 60 to 90-fold lower, including values and ranges therebetween, compared to the starting whole blood sample. In some embodiments, the RBC count of the PRP of the present disclosure is about 60 to 75-fold, about 60 to 70-fold, about 65 to 80-fold, about 65 to 70-fold, about 65 to 75-fold, about 70 to 80-fold, or about 75 to 80-fold lower, and so on, including values and ranges therebetween, compared to the starting whole blood sample. In some embodiments, the RBC count of the PRP of the present disclosure is about 60, 65, 70, 75, 80, 85 or 90-fold lower, including values and ranges therebetween, compared to the starting whole blood sample. In an exemplary embodiment, if the starting whole blood sample of a subject comprises about 4.7×10⁶ RBCs per microliter, the PRP prepared according to the present disclosure comprises about 0.06×10⁶ RBCs per microliter, which is about 78.3-fold reduction in RBCS than the starting whole blood sample. In another exemplary embodiment, for a whole blood sample of a subject comprising about 5.5×10⁶ RBCs per microliter, the PRP of the present disclosure comprises RBCs in the range of about 0.09 to 0.068×10⁶ per microliter, which is about 60 to 90-fold lower than the starting whole blood sample.

In some embodiments, the RBC count of the PRP of the present disclosure is about 145 to 155-fold, including values and ranges therebetween, reduced compared to the RBC count of the conventional PRP prepared using a single spin method. In some embodiments, the RBC count of the PRP of the present disclosure is about 145 to 150-fold, including values and ranges therebetween, lower than that of the conventional PRP prepared using the single spin method. In some embodiments, the RBC count of the PRP of the present disclosure is about 15 to 25-fold, or about 15 to 20-fold, or about 18 to 22-fold, including values and ranges therebetween, lower than the RBC count of the conventional PRP prepared using a double spin method.

In some embodiments, the WBC count of the PRP of the present disclosure is about 10 to 90-fold lower, including values and ranges therebetween, compared to the starting whole blood sample. In some embodiments, the WBC count of the PRP of the present disclosure is about 10 to 90-fold, about 10 to 25-fold, about 10 to 20-fold, about 15 to 30-fold, about 20 to 30-fold, or about 22 to 28, or about 28 to 30, or about 30 to 40, or about 40 to 50, or about 50 to 60 or about 60 to 70, or about 70 to 80 or about 80 to 90-fold lower, including values and ranges therebetween, compared to the starting whole blood sample. In some embodiments, the WBC count of the PRP of the present disclosure is about 10, 11, 12, 13, 14, 15, 16, 17, 18, 19, 20, 21, 22, 23, 24, 25, 26, 27, 28, 29, 30, 31, 32, and so on-fold lower, including values and ranges therebetween, compared to the starting whole blood sample. In an exemplary embodiment, if the starting whole blood sample of a subject comprises about 4.5×10³ WBCs per microliter, the PRP prepared according to the present disclosure comprises about 0.19×10³ WBCs per microliter, which is about 23.6-fold reduction in WBCs than the starting whole blood sample. In another exemplary embodiment, for a whole blood sample of a subject comprising about 6.5×10³ WBCs per microliter, the PRP of the present disclosure comprises WBCs in the range of about 0.65 to 0.216×10³ per microliter, which is about 10 to 90-fold lower than the starting whole blood sample.

In some embodiments, the WBC count of the PRP of the present disclosure is about 50 to 70-fold, about 55 to 65 fold, or about 55 to 70-fold, including values and ranges therebetween, reduced compared to the WBC count of the conventional PRP. In some embodiments, the WBC count of the PRP of the present disclosure is about 60, 61, 62, 63, 64, 65, 66, 67, 68, 69, or 70-fold, or about 60 to 70-fold, including values and ranges therebetween, lower than that of the conventional PRP prepared using the single spin method. In some embodiments, the WBC count of the PRP of the present disclosure is about 55, 56, 57, 58, 59, 60, 61, 62, 63, 64, or 65-fold, or about 55 to 65-fold, including values and ranges therebetween, lower than the WBC count of the conventional PRP prepared using a double spin method.

In some embodiments, the PRP of the present disclosure comprises about 1500-6750×10³ platelets per microliter, including values and ranges therebetween; about 0.05-0.1×10⁶ RBCS per microliter, including values and ranges therebetween; and/or about 0.1-0.45×10³ WBCs per microliter, including values and ranges therebetween.

In some embodiments, even if the platelet count of the PRP of the present disclosure is marginally higher or closer or may overlap with the platelet count of the conventional PRP; the RBC and/or the WBC count of the PRP of the present disclosure are substantially lower than those of the conventional PRP. In other words, the present PRP has substantially more fold reduction in the RBC count and/or the WBC count than the conventional PRP.

The present disclosure contemplates that the PRP can have any one of the cell counts, fold increase, and fold decrease features described herein, or a combination thereof. For example, in one embodiment, the PRP comprises a platelet count that is about 10 to 20-fold greater, including values and ranges therebetween, than starting whole blood sample. In another exemplary embodiment, the PRP comprises a platelet count that is about 10 to 20-fold greater, including values and ranges therebetween, and a RBC count that is 60 to 90-fold lower, including values and ranges therebetween, than starting whole blood sample. In another exemplary embodiment, the PRP comprises a platelet count that is about 10 to 20-fold greater, including values and ranges therebetween, than starting whole blood sample and a WBC count that is 10 to 90-fold lower, including values and ranges therebetween, than starting whole blood sample from same subject. In another embodiment, the PRP comprises a platelet count that is about 10 to 20-fold greater, including values and ranges therebetween; a RBC count that is 60 to 90-fold lower, including values and ranges therebetween; and a WBC count that is 10 to 90-fold lower, including values and ranges therebetween, than starting whole blood sample from same subject.

As the compositions herein, in some embodiments, comprise the GFC instead of the PRP, in order for the said composition to be manufactured, the present disclosure also provides a method for preparing the GFC from the convention PRP or the PRP of the present disclosure. Accordingly, the present disclosure also relates to a method for preparing a growth factor concentrate (GFC) obtained from the PRP prepared according to the methods described herein. That is, in some embodiments, the platelet-derived growth factor concentrate of the present disclosure is prepared from a PRP, wherein the PRP has a platelet count that is about 10 to 20-fold greater than starting whole blood sample, or a RBC count that is about 60 to 90-fold lower than starting whole blood sample, and/or a WBC count that is about 10 to 90-fold lower than starting whole blood sample.

While the GFC of the present disclosure is prepared from the PRP of the present disclosure, the method for preparing which is described herein, it will be understood by a person skilled in the art that similar steps can be applied to conventional PRP for obtaining GFC therefrom, To prepare the GFC, platelets present in the PRP are activated by subjecting the PRP to one or more platelet-activating treatments.

The GFC of the present disclosure is prepared from the PRP of the present disclosure. The methods for preparing the PRP of the present disclosure are described herein. To prepare the GFC, platelets present in the PRP are activated by subjecting the PRP to one or more platelet-activating treatments.

The platelet-activating treatment is selected from a group comprising treatment with platelet activation buffer and free-thaw cycles or a combination thereof.

In some embodiments, the platelet activation buffer comprises platelet activating agent selected from a group comprising collagen, calcium salt, hyaluronic acid, thrombin, and any combination thereof. In exemplary embodiments, the platelet-activating treatment comprises a combination of treatment with platelet activation buffer and one or more freeze-thaw cycles. In some embodiments, the PRP is treated with platelet activation buffer and said treated. PRP is subsequently subjected to one or more freeze-thaw cycles.

In some embodiments, the platelet activating agents such as collagen, a calcium salt, hyaluronic acid, thrombin, or a combination thereof are provided in a physiologically suitable buffer. In some embodiments, the platelet activating treatment comprises incubating the PRP, for about 15-45 minutes, with a buffer comprising collagen, a calcium salt, and hyaluronic acid. In some embodiments, the platelet activating treatment comprises incubating PRP, for about 15-45 minutes, with a buffer comprising collagen, hyaluronic acid, and thrombin. In some embodiments, the platelet activating treatment comprises incubating PRP, for about 15-45 minutes, with a buffer comprising a calcium salt, hyaluronic acid, and thrombin. In some embodiments, the platelet activating treatment comprises incubating PRP, for about 15-45 minutes, with a buffer comprising a calcium salt and hyaluronic acid followed by subjecting the PRP to freeze-thaw cycles. In some embodiments, the platelet activating treatment comprises incubating PRP, for about 15-45 minutes, with a buffer comprising collagen and hyaluronic acid followed by subjecting the PRP to freeze-thaw cycles. In some embodiments, the platelet activating treatment comprises incubating PRP, for about 15-45 minutes, with a buffer comprising thrombin and hyaluronic acid followed by subjecting the PRP to freeze-thaw cycles. In some embodiments, the platelet activating treatment comprises incubating PRP, for about 15-45 minutes, with a buffer comprising a calcium salt and thrombin followed by subjecting the PRP to freeze-thaw cycles. In some embodiments, about 10% to 30% by volume of a buffer containing platelet-activating agents is added to PRP. For example, about 100 microliter of the buffer containing platelet-activating agents is added to 1 ml of PRP.

In some embodiments, the freeze thaw cycle(s) can be carried out prior to or along with or followed by the treatment with the buffer. In some examples, the order of the freeze thaw cycle(s) does not impact the processing of the PRP.

In some embodiments, the PRP incubated with a buffer containing platelet-activating agents is subjected to 2-7 freeze-thaw cycles. A freeze-thaw cycle comprises freezing the PRP incubated with one or more platelet-activating agents to about 4° C., −20° C., or −80° C., and thawing the frozen PRP at a temperature of about 20° C. to 37° C. or about 25° C. to 37° C. The PRP upon treatment with a platelet-activating treatment forms a gel-like consistency (FIG. 7). The gel upon standing separates spontaneously from liquid supernatant. The supernatant contains the GFC.

In some embodiments, the method for preparing GFC comprises: (a) incubating a whole blood sample collected in an anti-coagulant container with RBC aggregating agent(s); (b) subjecting the whole blood sample incubated with the RBC aggregating agent to a first centrifugation step to obtain a supernatant containing platelets; (c) subjecting the supernatant to a second centrifugation step to obtain a platelet pellet and platelet-poor plasma (PPP); and (d) resuspending the platelet pellet in PPP to obtain the PRP; (e) subjecting the PRP to platelet-activating treatment; and (f) collecting supernatant containing the growth factor concentrate.

In some embodiments, the method for preparing GFC comprises (a) incubating a whole blood sample collected in an anti-coagulant container with RBC aggregating agent(s) selected from a group comprising heparin, collagen, a calcium salt, hyaluronic acid, polygeline, thrombin, gelatin, EDTA, sodium citrate, starch, and any combination thereof, wherein the incubation is carried out at a temperature of about 20-25° C.; (b) subjecting the whole blood sample incubated with the RBC aggregating agent to a first centrifugation step to obtain a supernatant containing platelets, wherein the first centrifugation is carried out at about 300-1000 rpm for about 2-10 minutes; (c) subjecting the supernatant to a second centrifugation step to obtain a platelet pellet and platelet-poor plasma (PPP), wherein the second centrifugation is carried out at about 1200-3500 rpm for about 5-15 minutes; and (d) resuspending the platelet pellet in PPP to obtain the PRP (e) activating platelets in the PRP by subjecting the PRP to a platelet-activating treatment selected from a group comprising treatment with platelet activation buffer and free-thaw cycles or a combination thereof, wherein the platelet activation buffer comprises platelet activating agent selected from a group comprising collagen, a calcium salt, hyaluronic acid, thrombin, and any combination thereof; and (f) collecting supernatant containing the growth factor concentrate.

Once obtained, the platelet-derived growth factor concentrate (GFC) can be put to application instantly or may be subjected to storage for subsequent use. In a non-limiting embodiment, the GFC is stored in air tight vials. Storage without diminished quality is feasible for a period of about 6 months, at a storage temperature ranging from about −196° C. to +4° C.

In some embodiments, the PRP or the GFC of the present disclosure comprise peripheral blood stem cells (PBSCs), at a concentration ranging from about 10% to 50%. This is another composition that can be therapeutically employed for the treatment of azoospermia as per the present disclosure.

Throughout, this disclosure, if the concentration of PRP/GFC is expressed in terms of percentages, it refer to the volume of PRP/GFC added to the composition—e.g., 30% PRP/GFC means 300 μl of PRP/GFC is added to make 1 ml of the composition or 3 ml of PRP/GFC is added to make 10 ml of the composition. Similarly, throughout this disclosure, if the concentration of PBSCs is expressed in terms of percentages, it refer to the volume of PBSC solution added to the composition—e.g., 40% PBSCs means 4 ml of PBSC solution is added to make 10 ml of the composition.

The above described order of steps is not binding on the method of the present disclosure and does not restrict the order in which the steps must be performed. The steps may be performed in any order that is logically feasible. The possibility of supplementing the methods of the present disclosure with steps/modifications routinely practiced in the art in relation to preparation of PRP and platelet derived growth factor compositions is envisaged by the present disclosure.

Accordingly, in some embodiments of the present disclosure, the polymer is the last component to be included in the compositions herein, prior to administration to the subject. Thus, for example when PBSCs and/or additional therapeutic agents are included in the composition, they are first mixed with the PRP or the GFC and then the combination is mixed with polymer just prior to the final administration.

Now, in order to facilitate preparation of the PRP or the GFC of the present disclosure, and subsequently the compositions herein, the present disclosure also provides a kit.

Thus, the present disclosure provides a kit for preparing the therapeutic compositions of the present disclosure, wherein the kit as comprises:
a) G-CSF;
b) a RBC activating agent selected from a group comprising: heparin, collagen, a calcium salt, hyaluronic acid, polygeline, thrombin, gelatin, EDTA, sodium citrate, starch, and a combination thereof;
c) a thermoresponsive polymer; and
d) an instruction manual.

In some embodiments, the kit of the present disclosure further comprises a platelet activating agent selected from a group comprising: collagen, a calcium salt, hyaluronic acid, and thrombin, or a combination thereof. The kit also comprises a blood collection container comprising an anti-coagulant.

In some embodiments, the kit of the present disclosure further comprises an additional therapeutic agent selected from a group comprising hormone, growth factor, protein, cell, cell secretome, and drug, or any combination thereof; and wherein the agent is selected from a group comprising follicle stimulating hormone (FSH), luteinizing hormone (LH), high-density lipoprotein (HDL), steroidogenic acute regulatory protein (StAR), stem cell, phosphodiesterase V inhibitors, sildenafil sitrate, 1 adrenergic blocker and alprostadil, or any combination thereof.

As is clear, the kit of the present disclosure is used for preparing the therapeutic compositions herein. In other words, the kit of the present disclosure allows for:
a) processing of whole blood for preparation of PRP of the present disclosure;
b) processing of whole blood for preparation of GFC from the PRP of the present disclosure;
c) processing of conventional PRP for preparation of GFC of the present disclosure;
d) preparing of the therapeutic compositions of the present disclosure comprising PRP and thermosensitive polymer; and/or
e) preparing of the therapeutic compositions of the present disclosure comprising GFC and thermosensitive polymer.

Since the kit comprises the RBC activating agent, in some embodiments, the kit also facilitates preparation of PBSCs for inclusion in the compositions of the present disclosure. Accordingly, the kit of the present disclosure also allows for:
a) preparing of the therapeutic compositions of the present disclosure comprising PRP and thermosensitive polymer, and PBSCs; and
b) preparing of the therapeutic compositions of the present disclosure comprising GFC and thermosensitive polymer, and PBSCs.

Further, since the kit comprises one or more additional therapeutic agent, in some embodiments, the kit also facilitates preparation of the compositions of the present disclosure having said additional therapeutic agent.

In some embodiments, the kit comprises an instruction manual having steps for: processing of the whole blood for processing of whole blood for preparation of PRP of the present disclosure; processing of whole blood for preparation of GFC from the PRP of the present disclosure; processing of conventional PRP for preparation of GFC of the present disclosure; preparing of the therapeutic compositions of the present disclosure comprising PRP and thermosensitive polymer; and/or preparing of the therapeutic compositions of the present disclosure comprising GFC and thermosensitive polymer. The instructional manual may additionally comprise steps for processing of PBSCs and/or inclusion on additional therapeutic agent during preparation of any of the said compositions.

It is to be understood by a person skilled in the art that the embodiments relating to the use of the kit on possibilities of processing the blood, and/or preparing the compositions herein are only exemplary in nature, and all possible permutations-combinations that are possible within the ambit of the present disclosure are equally applicable to the use of the kit, as long as the kit is able to facilitate the said processing or preparation.

Once the compositions of the present disclosure are prepared as outlined herein, they are used for treating infertility in men, caused by azoospermia. Accordingly, the present disclosure relates to a method for treating azoospermia in a subject in need thereof comprising, administering to the subject the therapeutic compositions of the present disclosure.

In some embodiments, the therapeutic composition is administered to one or both testis of the subject; and wherein the administration is repeated for at least one or more times.

In some embodiments, the therapeutic composition is administered to each testis in an amount ranging from about 0.5 ml to about 1.5 ml. Accordingly, the therapeutic composition is administered to each testis in an amount of about 0.6 ml, 0.7 ml; 0.8 ml, 0.9 ml, 1 ml; 1.1 ml, 1.2 ml, 0.3 ml, 1.4 ml or 1.5 ml.

In some embodiments, the administration to each testis is repeated 1 to 5 times, with a gap of 3 months, over a total period of about 3 to 15 months. In other words, the composition is administered every 3 months, depending on the severity of the azoospermia and the need based on the subject. For example, the composition is administered on day 0 (first administration), then in 3^{rd} month (second administration), then again in 6^{th} month (third administration), and so on, depending on the clinical parameters, and need of continued treatment.

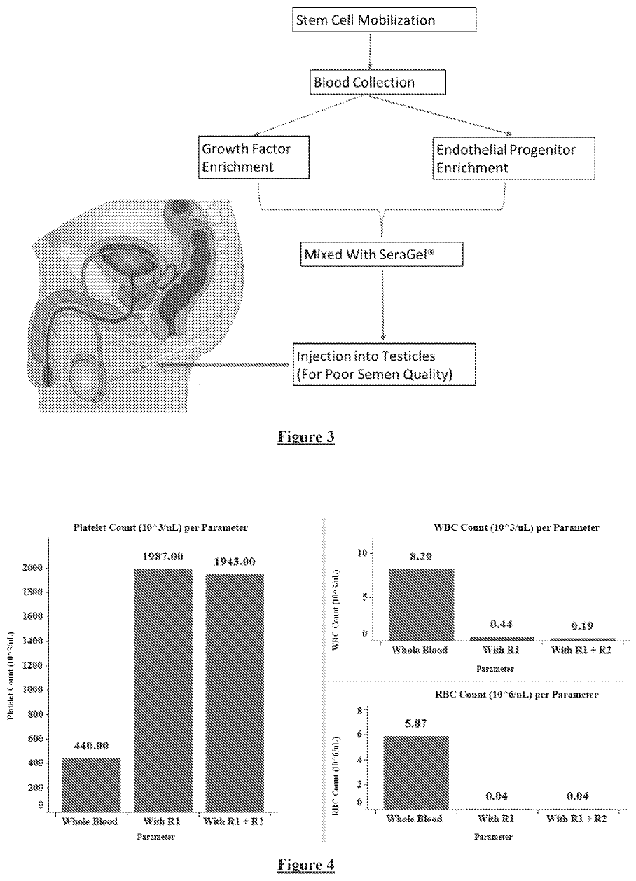

As the compositions of the present disclosure comprise of PRP or the growth factor concentrate, the underlying growth factors present therein help in the treatment due to its well-known regenerative potential. When PRP or PRP derived GFC is administered to the testis, the growth factors excite the sperm cells and existing dormant growth factors in the testicles. The growth factors and cytokines turn the sperms motile, increase the sperm count and quality, decrease malformation and increase viability of the sperm. This way, the compositions herein make the semen potentially more fertile and increases the fertilization capacity. As an exemplary representation, the schematic scheme for preparing the composition of the present disclosure and the subsequent administration is provided in FIG. 3.

As the present disclosure contemplate inclusion of the PBSCs in the compositions herein, in some embodiments, the subject is administered G-CSF for a period of one to three days prior to the administration of the composition of the present disclosure. Accordingly, in some embodiments, on the day of the treatment by administration of the composition, the following process is performed:
a) about 30 ml of whole blood is withdrawn followed by optional segregation into two fractions—one for preparing the composition and another for preparing the solution containing the PBSCs. Alternatively, two separate fractions can be withdrawn from the subject for the two activities;
b) employing the first fraction to prepare the composition of the present disclosure, and the second fraction for preparing the concentrated solution containing the PBSCs;
c) mixing of the prepared composition with the concentrated solution of the PBSCs to arrive at the final composition for administering to the subject in need of treatment for azoospermia.

In some embodiments, the total amount of blood that is withdrawn from the subject ranges from about 10 ml to 60 ml, depending on the amount of final composition that needs to be prepared for administration. A person skilled in the art would readily understand, based on the need for treatment of azoospermia, varying from very mild to mild to severe to very severe condition. Based on the information provided in the present disclosure, and depending on the final amount of the composition that will be administered (between 0.5 ml to 1.5 ml per testis), and depending on the concentrations of the components therein—the PRP or GFC, the polymer and optionally the PBSCs and/or additional therapeutic agents, appropriate amount of blood is withdrawn.

In some embodiments, since the preparations of the compositions of the present disclosure can be carried out with allogenic blood, the amount of blood that is then employed for preparation of the final composition, also remains identical to what is described in the previous embodiment.

The detailed steps involved in preparation of the composition as mentioned in the preceding embodiment, along with preparation of the solution containing PBSCs are as per the methods provided in the present disclosure.

In alternative embodiments, in case no PBSCs are included in the final composition, the step of G-CSF administration and preparation of the solution containing PBSCs is eliminated.

Since the compositions of the present disclosure comprise a thermoresponsive polymer, it is to be noted that while the composition will be in a liquid form during the preparation and administration, owing to its temperature sensitive nature, the composition comprising the thermoresponsive polymer will convert into a gel form upon contact with physiological temperature. This will allow the composition to be retained by the testis, and avoid dilution of the delivered material and result in sustained localised delivery of the composition.

Upon administration, parameters including quality and quantity of sperm per ejaculation, volume of semen, motility of sperm, morphological shape, testicular size, structural improvements and sexual potency are observed. Also, evaluation of increase in testosterone levels and decrease in FSH, LH and Prolactin Levels is evaluated for understanding the effect of the treatment in men with azoospermia.

While the instant disclosure is susceptible to various modifications and alternative forms, specific aspects thereof have been shown by way of examples and drawings and are described in detail below. However, it should be understood that it is not intended to limit the invention to the particular forms disclosed, but on the contrary, the intention is to cover all modifications, equivalents, and alternatives falling within the spirit and the scope of the invention as defined by the appended claims.

### EXAMPLES

### Example 1: Preparation of Platelet-Rich Plasma (PRP)

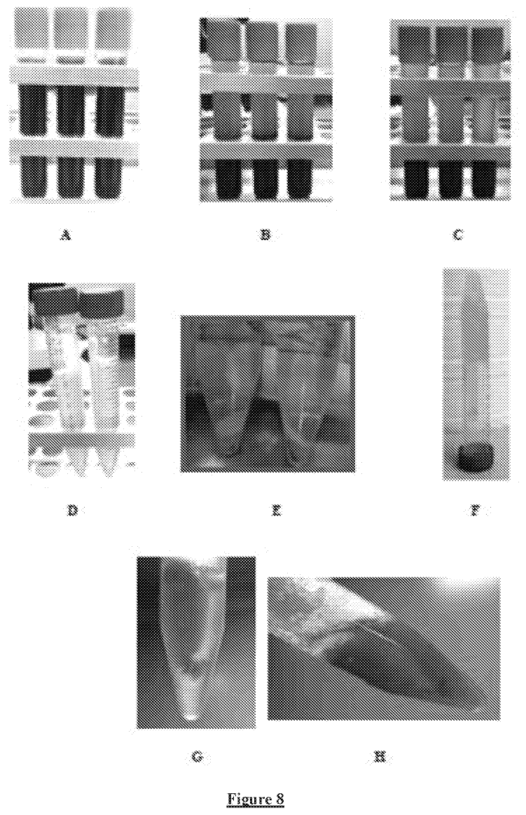

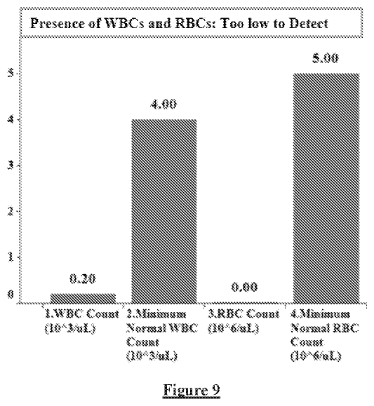

A 30 ml of venous blood was drawn from a patient and 10 ml each was aliquoted into acid citrate dextrose (ACD-A) solution gel tube/K2 EDTA tube. The samples were incubated for 45 minutes with a buffer comprising polygeline, gelatin, and starch as RBC aggregating agents (FIG. 9). After incubation, samples were centrifuged at 600 rpm for 2 minutes. Supernatant containing platelets was collected and again centrifuged at 3000 rpm for 12 minutes. After this centrifugation, platelets sedimented as a pellet and the supernatant contained platelet-poor plasma (PPP). The platelet pellet was resuspended in 3 ml of PPP to obtain PRP (FIG. 8).

The number of platelets, RBCs, and WBCs in the PRP were counted. The table 2 below shows the cell count obtained by the above-described method (PRP of the present disclosure) and comparative cell count obtained by conventional PRP methods. The cell count values for conventional PRP methods are based on the values disclosed in prior art, for example in Principles and Methods of Preparation of Platelet-Rich Plasma: A Review and Author's Perspective (J Cuian Aesthet Surg. 2014 October-December; 7(4): 189-197.doi: 10.4103/0974-2077)

TABLE 2

| | | Platelets | | | |
|---|---|---|---|---|---|
| | | | Fold increase | Total WBC | RBC |
| | | Count | over whole | Count | Count |
| Parameters | | 10\hat{ }3/ul | blood | 10\hat{ }3/ul) | (10\hat{ }6/ul) |
| 1 | Whole Blood (Minimum | 150 | — | 4.5 | 4.7 |
| | Normal Value) | | | | |
| 2 | Conventional PRP | 1096 | 7.4 | 12.6 | 8.9 |
| | Protocol (Single | | | | |
| | Spin/Buffy Coat Method) | | | | |
| 2 | Conventional PRP Protocol | 1577 | 10.5 | 11.3 | 1.1 |
| | (Double Spin/PRP method) | | | | |
| 3 | PRP Method of the present | 2023 | 13.4 (1.8 fold | 0.19 (23.6 fold | 0.06 (78.33 fold |
| | disclosure | | over single | reduction over | reduction over |
| | | | spin/1.3 fold | whole blood/66.3 | whole blood/148.3 |
| | | | over double spin) | fold reduction over | fold reduction over |
| | | | | single spin/59.47 | single spin/18.33 |
| | | | | fold reduction | fold reduction |
| | | | | over double spin) | over double spin) |

### Example 2: Preparation of Platelet-Derived Growth Factor Concentrate (GFC)

PRP was prepared as described in Example 1. 300 μl of a platelet activation buffer comprising calcium chloride and thrombin was mixed with the PRP and the mixture was incubated for 45 minutes. After incubation, the mixture was subjected to three freeze-thaw cycles with freezing at 4° C. and thawing at 37° C. The supernatant containing the GFC was collected and aliquoted into cryovials, which can be used for administration right away or can be preserved for future use (FIG. 8).

ELISA assays were performed to determine levels of growth factors present in the freshly-prepared GFC and the levels upon storage at 20° C. or −10° C. The table 3 below shows the levels in the freshly-prepared GFC and the levels upon storage at 20° C. for a duration of 3, 6, 9, and 12 hours.

TABLE 3
Freshly-prepared and upon storage at 20° C

| | | pg/ml | pg/ml | pg/ml | ng/ml | ng/ml | ng/ml |
|---|---|---|---|---|---|---|---|
| Duration | | VEGF | EGF | bFGF | IGF-1 | PDGF-BB | TGF-b1 |
| Fresh | | 914 ± 400 | 183 ± 50 | 50.2 ± 24.0 | 102.7 ± 26.5 | 53.2 ± 32.3 | 294 ± 45.2 |
| 1 | h | 901 ± 390 | 190.2 ± 34.2 | 54 ± 22.7 | 108.5 ± 28.4 | 60.2 ± 22.4 | 310.2 ± 34.2 |
| 3 | h | 850.2 ± 381.2 | 178 ± 43.2 | 47 ± 21.4 | 98.7 ± 26.5 | 57 ± 21.4 | 280 ± 48.2 |
| 6 | h | 839.1 ± 390.6 | 160 ± 46.2 | 45 ± 23.5 | 93.7 ± 25.5 | 43 ± 27.5 | 290 ± 46.2 |
| 9 | h | 222.4 ± 45.3 | 65 ± 22.4 | 19 ± 10.5 | 22.3 ± 18.2 | 21 ± 11.5 | 135 ± 23.4 |
| 12 | h | 112 ± 45.3 | 46 ± 20.4 | 18 ± 23.5 | 24.4 ± 17.5 | 14 ± 13.5 | 60.2 ± 22.4 |

The table 4 below shows the levels in the freshly-prepared GFC and the levels upon storage at −10° C. for a duration of 1 week, 4 weeks, 8 weeks, 12 weeks and 24 weeks.

TABLE 4
Freshly-prepared and upon storage at −10° C.

| | | VEGF | EGF | bFGF | IGF-1 | PDGF-BB | TGF-b1 |
|---|---|---|---|---|---|---|---|
| Duration | | pg/ml | pg/ml | pg/ml | ng/ml | ng/ml | ng/ml |
| Fresh | | 914 | 183 | 50.2 | 102.7 | 53.2 | 294 |
| 1 | w | 890 | 190 | 58 | 110 | 62 | 260 |
| 4 | w | 850.2 | 210 | 51 | 97 | 56 | 280 |
| 8 | w | 839.1 | 170 | 47 | 93.7 | 43 | 290 |
| 12 | w | 890 | 200 | 50 | 82 | 49 | 240 |
| 24 | w | 860 | 160 | 46 | 96 | 51 | 270 |

### Example 3: Preparation of Peripheral Blood Stem Cells (PBSCs)

A 10 ml of venous blood was drawn from a patient into an acid citrate dextrose (ACD-A) solution gel tube/K2 EDTA tube. The sample was incubated for 45 minutes with a buffer comprising polygeline, gelatin, and starch as RBC aggregating agents. After incubation, samples were centrifuged at 1500 rpm for 10 minutes. Upon centrifugation, RBCs, WBCs, and platelets were separated as follows: the bottom layer contained RBCs, the middle layer contained platelets and WBCs (buffy coat layer) and the top layer was platelet-poor plasma. The top layer (PPP) was removed and the middle buffy coat layer was transferred to another sterile tube. The tube was centrifuge at 2000 rpm for 12 minutes to separate WBCs. Alternatively, leucocyte filtration filter can be used to separate WBCs. The table 5 below shows the WBC, RBC, and platelet count of the PBSC solution obtained using this method. The numbers in parenthesis in the last column indicate fold increase over whole blood.

TABLE 5
Cell count of PBSCs

| Parameters | Whole blood (Range) | Buffy coat/PBSCs | |
|---|---|---|---|
| WBC(×10\hat{ }3/ul) | 1.44-30.75 | 5 | (5x) |
| RBC (×10\hat{ }6/ul) | 1.66-5.96 | 1.0 | |
| PLT (×10\hat{ }3/ul) | 150-450 | 690 | (>4x) |

### Example 4: Analysis of the Effect of RBC Aggregators on the PRP Profile

Example 1 was repeated with the following variations—
A) Employment of a single RBC aggregator—Gelatin
B) Employment of a combination of 2 RBC aggregators—Gelatin and Starch
C) Employment of no RBC aggregator
D)-F) No RBC aggregators

Experiments A-F were designed to have gradually increasing centrifugation speed and time (to compensate for absence of the RBC aggregators, especially for experiments D-F). G was a control experiment.

Specifics of the above experiments are depicted in table 6 below.

TABLE 6
Blood Processing for PRP - Protocol Standardization

| Step | Parameter | A | B | C | D | E | F | G |
|---|---|---|---|---|---|---|---|---|
| 1 | RBC | With RBC1 | With RBC1 + 2 | Whole Blood | | | | |
| | aggregators | | | | | | | |
| 2 | Incubation | 15 | 30 | 45 | No | | | |
| | time-minutes | | | | | | | |
| 3 | Centrifugation- | 500 | 600 | 700 | 800 | 900 | 1000 | No |
| | rpm | | | | | | | |
| 4 | Centrifugation- | 2 | 4 | 6 | 8 | 10 | | |
| | time-minutes | | | | | | | |
| 5 | Platelet | Ca Salt-45 mins | Thrombin-45 mins | Ca + Thrombin- | Freeze-Thaw | Freeze-Thaw | | |
| | activation | | | 45 mins | (4degree-37 | LN2 10 mins/ | | |
| | | | | | degree/10 | cyclex3 | | |
| | | | | | mins/cyclex3 | | | |

| | | |
|---|---|---|
| 4 | GFC assay- ELISA | 9*5 Assays |

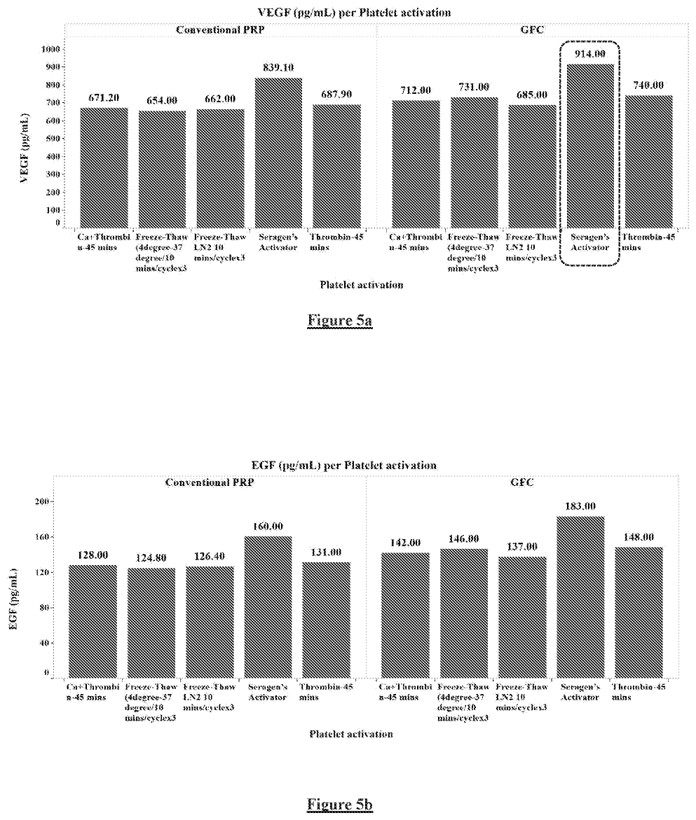

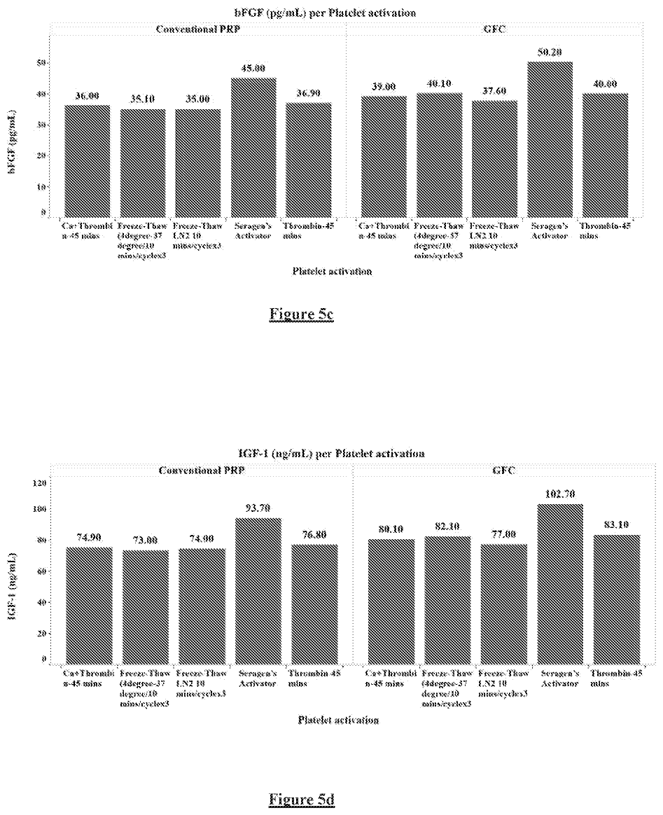

Experiments A and B which employed RBC aggregators were found to yield improved results with respect to settling and separation of RBCs and WBCs through their respective protocols. For reasons of brevity, results from variations of the experiment closest to the protocol of the present disclosure are depicted as graphs in FIG. 4. As can be observed from said figure, the incorporation of RBC aggregators in the PRP/GFC preparation protocol has a significant impact in terms of the improvement in platelet count and reduction in RBC and WBC count. The combination of 2 RBC aggregators was found to further improve the reduction in WBC count in the PRP.

### Example 5: Analysis of the Effect of Different Platelet Activation Protocols

The effect of the choice of platelet activation protocol on the concentration of growth factors in the final GFC was analyzed by performing variations of the experiment in Example 2. Keeping other specifics of the experiment constant, said variations employed treatment of PRP with single platelet activating agent, treatment of PRP with a combination of 2 activating agents, exposure of PRP to freeze-thaw cycles at different temperatures and a combination of treatment of PRP with activation agent and exposure to freeze-thaw cycles.

Results yielded by said experiments are provided in the table 7 below.

TABLE 7

| | | VEGF | EGF | bFGF | IGF-1 | PDGF-BB | TGF-b1 |
|---|---|---|---|---|---|---|---|
| Platelet activation | | (pg/ml) | (pg/ml) | (pg/ml) | (ng/ml) | (ng/ml) | (ng/ml) |
| PRP of | Activation buffer - | 740 ± 380 | 148 ± 30 | 40 ± 22 | 83.1 ± 23 | 43 ± 28.9 | 238 ± 35.6 |
| the present | Thrombin-45 mins | | | | | | |
| disclosure | Activation buffer - | 712 ± 395 | 142 ± 42 | 39 ± 19 | 80.1 ± 19.2 | 41 ± 21.3 | 229 ± 31.2 |
| | Ca + Thrombin-45 mins | | | | | | |
| | Freeze-Thaw | 731 ± 372 | 146 ± 51 | 40.1 ± 15 | 82.1 ± 14.3 | 42.5 ± 18.7 | 235 ± 29 |
| | (4degree-37 degree/ | | | | | | |
| | 10 mins/cyclex3 | | | | | | |
| | Freeze-Thaw LN2 10 | 685 ± 437 | 137 ± 40 | 37.6 ± 10 | 77 ± 21.1 | 39.9 ± 19.5 | 220 ± 25 |
| | mins/cyclex3 (−196° C.) | | | | | | |
| | Activation with Calcium | 914 ± 400 | 183 ± 50 | 50.2 ± 24.0 | 102.7 ± 26.5 | 53.2 ± 32.3 | 294 ± 45.2 |
| | buffer + Freeze- | | | | | | |
| | Thaw cycles | | | | | | |
| Conventional PRP | Thrombin-45 mins | 687.9 ± 41.3 | 131 ± 41.3 | 36.9 ± 19.4 | 76.8 ± 24.4 | 35 ± 23.4 | 237.8 ± 41.2 |
| | Ca + Thrombin- | 671.2 ± 362 | 128 ± 43.2 | 36 ± 18.9 | 74.9 ± 19.2 | 34.4 ± 18.3 | 232 ± 38.7 |
| | 45 mins | | | | | | |
| | Freeze-Thaw | 654 ± 358 | 124.8 ± 35.2 | 35.1 ± 21.1 | 73 ± 14.4 | 33.5 ± 19.6 | 226.2 ± 39.2 |
| | (4degree-37 degree/ | | | | | | |
| | 10 mins/cyclex3 | | | | | | |
| | Freeze-Thaw LN2 10 | 662 ± 379 | 126.4 ± 39.1 | 35.55 ± 17.3 | 74 ± 19.5 | 33.9 ± 16.2 | 229.1 ± 42 |
| | mins/cyclex3 | | | | | | |
| | Activation with | 839.1 ± 390.6 | 160 ± 46.2 | 45 ± 23.5 | 93.7 ± 25.5 | 43 ± 27.5 | 290 ± 46.2 |
| | Calcium buffer + | | | | | | |
| | Freeze-Thaw cycles | | | | | | |

Cyclex 3 process—Between freezing and thawing: 1. Sample will be frozen and kept as frozen for 10 minutes; 2. Sample will be thawed and kept as it is for 10 mins; and 3. Step 1&2 will be repeated three times.

Observations from the above experiments show that a platelet activation protocol employing a combination of treatment of PRP with activation agent and exposure of PRP to freeze-thaw cycles yields GFC with significantly higher growth factor concentration—said effect being observed for both the PRP of the present disclosure as well as conventional PRP.

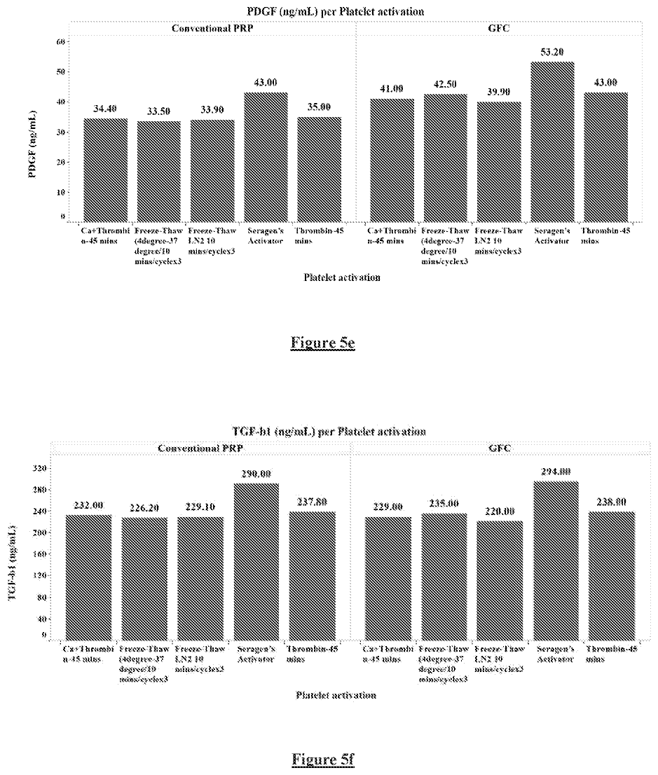

The PRP of the present disclosure, however, provides a notably higher concentration of individual growth factors in the GFC derived therefrom when compared to conventional PRP that is subjected to platelet activation by the same protocol. Thus, a synergy between the PRP preparation protocol and PRP activation protocol in yielding GFC with high growth factor concentration is derivable from the above data. The above results are depicted in FIG. 5.

### Example 6: Preparation of Composition Comprising PRP and Thermoresponsive Polymer

For preparing a composition comprising PRP and thermoresponsive polymer [(NIPAM based polymer-poly(Nisopropylactylamide-co-n-butyl methacrylate) poly(NIPAAm-co-BMA)], the first step was to obtain the PRP. As described in the present disclosure, the PRP can either be obtained from whole blood by conventionally known methods, or by specific protocol as recited in example 1 above.

In the present example, the objective was to prepare 1 ml of the composition for administration into testis of an azoospermic subject. Accordingly, about 0.5 ml of the PRP prepared by the exemplified protocol was taken for mixing with 0.5 ml or 50% (as a final concentration) of the thermoresponsive polymer.

Separately, the thermoresponsive polymer, which was in the form of a powder, was subjected to mixing with water or saline to form a solution having a concentration of about 50%. For this, the following steps were performed:
a) the thermoresponsive polymer was dissolved in water to obtain a solution having up to about 50% w/w of polymer(s);
b) the solution was stirred at speed of 30-100 rpm at about 10° C. at for about 15 minutes; and
c) the mixture was rocked for about 15 minutes thereby forming a solution.

In an alternate experiment, the thermoresponsive polymer, was directly taken in the form of a powder for mixing with the PRP, without dissolution in water or saline.

Accordingly, two batches of mixtures were prepared. One comprising about 0.5 ml of the PRP and 0.5 ml of the solution of the polymer; and the second comprising about 1 ml of the PRP and the polymer powder (50%). For preparation of these mixtures, the following steps were performed:
a) the thermoresponsive polymer was contacted with the PRP in a sterile tube, and the mixture was cooled in refrigerator at a temperature of about 4° C. for about
b) the tube was periodically shaken to help mixing of the contents and maintained at the same temperature;
c) once dissolved, the mixture was allowed to settle for elimination of air bubbles.

This mixture comprised of 0.5 ml of PRP and 0.5 ml or 50% of the thermoresponsive polymer.

This experiment was subsequently repeated by replacing the NIPAM based polymer with Poloxamer 407 to obtain a composition comprising PRP and Poloxamer 407.

These final compositions were prepared for administration to an azoospermic subject.

### Example 7: Preparation of Composition Comprising GFC and Thermoresponsive Polymer

For preparing a composition comprising GFC and thermoresponsive polymer [(NIPAM based polymer-poly(Nisopropylacrylamide-co-n-butyl methacrylate) poly(NIPAAm-co-BMA)], the first step was to obtain the GFC. As described in the present disclosure, the GFC can either be obtained from conventionally known PRP; or by specific protocol as recited in example 2 above.

In the present example, the objective was to prepare 0.8 ml of the composition for administration into testis of an azoospermic subject. Accordingly, about 0.4 ml of the PRP prepared by the exemplified protocol was taken for mixing with 0.4 ml or 50% (as a final concentration) of the thermoresponsive polymer.

Separately, the thermoresponsive polymer, which was in the form of a powder, was subjected to mixing with water or saline to form a solution having a concentration of about 50%. For this, the following steps were performed:
a) the thermoresponsive polymer was dissolved in 50 ml of water to obtain a solution having up to about 50% w/w of polymer(s);
b) the solution was stirred at about 30-100 rpm at about 10° C. at for about 15 minutes; and
c) the mixture was rocked for about 15 minutes thereby forming a solution.

In an alternate experiment, the thermoresponsive polymer, was directly taken in the form of a powder for mixing with the GFC, without dissolution in water or saline.

Accordingly, two batches of mixtures were prepared. One comprising about 0.4 ml of the GFC and 0.4 ml of the solution of the polymer; and the second comprising about 0.4 ml of the GFC and 0.4 ml of the polymer or the powder sufficient for (50%). For preparation of these mixtures, the following steps were performed:
a) the thermoresponsive polymer was contacted with the PRP in a sterile tube, and the mixture was cooled in refrigerator at a temperature of about 8° C. for about 10 minutes;
b) the tube was periodically shaken to help mixing of the contents and maintained at the same temperature;
c) once dissolved, the mixture was allowed to settle for elimination of air bubbles.

This mixture comprised of 0.4 ml of PRP and 0.4 ml or 50% of the thermoresponsive polymer.

This experiment was subsequently repeated by replacing the NIPAM based polymer with Poloxamer 407 to obtain a composition comprising GFC and Poloxamer 407.

These final compositions were prepared for administration to an azoospermic subject.

### Example 8: Preparation of Composition Comprising PRP or GFC and Thermoresponsive Polymer Along with PBSCs

For preparing a composition comprising PRP or GFC and thermoresponsive polymer [(NIPAM based polymer-poly(Nisopropylacrylamide-co-n-butyl methacrylate) poly(NIPAAm-co-BMA)], along with PBSCs, the first step was to obtain the PRP or the GFC. As described in the present disclosure, the PRP can either be obtained from conventionally known PRP, or by specific protocol as recited in example 1 above. Similarly, the GFC can either be obtained from conventionally known PRP, or by specific protocol as recited in example 2 above.

In the present example, the objective was to prepare 1 ml of the composition for administration into testis of an azoospermic subject. Accordingly, about 0.30 ml of the PRP prepared by the exemplified protocol was taken for mixing with 0.20 ml or 20% (as a final concentration) of the thermoresponsive polymer. In an alternate experiment, about 0.30 ml of the GFC prepared by the exemplified protocol was taken for mixing with 0.20 ml or 20% (as a final concentration) of the thermoresponsive polymer.

Separately, four batches of the thermoresponsive polymer were prepared—two in solution form (similar to examples 6 and 7 above) and two directly in the powder form.

Separately, four fractions of 0.5 ml (50% of the final composition) of the PBSCs were prepared from the whole blood of the subject, as per the huffy coat protocol described in example 3 above.

For preparing the final compositions, four batches of initial mixtures were prepared, that comprised of PRP or GFC and PBSCs for final mixing with the polymer as follows:
1) PRP and PBSC for mixing with polymer in powder form,
2) PRP and PBSC for mixing with polymer in solution form;
3) GFC and PBSC for mixing with polymer in powder form; and
4) GFC and PBSC for mixing smith polymer in solution form.

Each of these batches comprised of about 0.30 ml of the PRP or GFC respectively and about 0.50 ml or 50% of the PBSCs. For preparation of these mixtures, simple mixing steps were carried out.

To these 4 batches, the 4 fractions of 0.20 ml (20%) of the polymer was added, to prepare the final composition for administration to an azoospermic subject. For preparation of these final mixtures, mixing steps similar to those in examples 6 and 7 were followed. The following table 8 provides for the particulars of the composition prepared herein;

TABLE 8

| | Testicles |
|---|---|
| Particulars | (per side) |
| Cells/Stem cells (V %) | 50 |
| GFC/PRP (V %) | 30 |
| Polymer (V %) | 20 |
| Final volume (ml) | 1 |

This experiment was subsequently repeated by replacing the NIPAM based polymer with Poloxamer 407 to obtain a composition comprising PRP or GFC, Poloxamer 407 and PBSCs.

### Example 9: Preparation of Composition Comprising PRP or GEC and Thermoresponsive Polymer with Additional Therapeutic Agent

For preparing a composition comprising PRP or GFC and thermoresponsive polymer [(NIPAM based polymer-poly(Nisopropylacrylamide-co-n-butyl methacrylate) poly(NIPAAm-co-BMA) or Poloxamer 407], with or without PBSCs, and with additional therapeutic agent, the overall protocol remained the same as those described in the previous examples. The inclusion of the additional therapeutic agent was affected at stage prior to mixing of the components with the polymer. The PRP or GFC was first mixed with the additional therapeutic agents, in this case all the additional growth factors, i.e., VEGF, NGF, FGF, HGF, I-IGF-I, EGF, PDGF, Transforming growth factor-b/family, and SGF, to make the final concentrations of the growth factors as provided in table 1 herein, Thereafter, the mixture was added with the polymer as per protocol of examples 6 and 7, depending on PRP or GFC as the first component.

### Example 10: Effect of Thermoresponsive Polymer on Release Profile of the Composition Comprising PRP or Recombinant Growth Factor

This example was designed for assessing the importance of the thermoresponsive polymer in the compositions of the present disclosure. This was carried out by comparing the growth factor release profile from a composition comprising the polymer, and a composition devoid of it. For further analysis on the effect of the polymer, regardless of the underlying active component, a test composition of recombinantly prepared VEGF with the polymer was also prepared.

In this example, composition comprising PRP and thermoresponsive polymer was prepared as per the protocol provided in example 6 above. To compare the effect of the said polymer, a preparation of PRP (as per the protocol of example 1 above) in equal volume of phosphate buffer saline was prepared. The test composition of recombinant VEGF with the polymer, was prepared by a simple 1:1 mixing of the recombinant VEGF with the polymer.

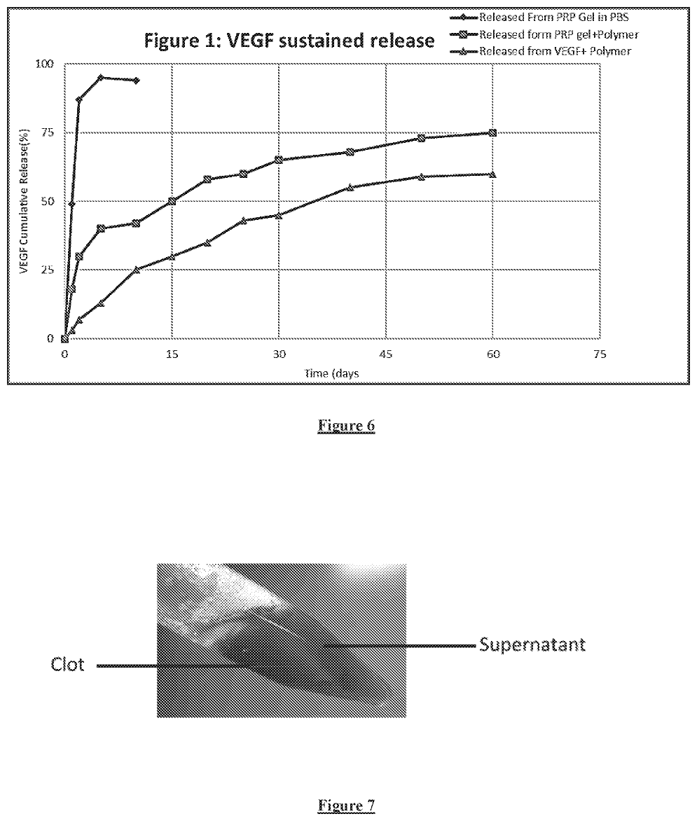

The in vitro growth factor release kinetics was performed in PBS (pH 7.4) at 37° C. for 60 days as reported in FIG. 6. As can be seen, VEGF released from PRP mixed with polymer within the first 2 days (burst effect) was 30±3%, followed by a phase of sustained release with almost 75% of VEGF being released within 60 days (orange/middle graph). Although, the VEGF release was lower for composition of recombinant VEGF mixed with polymer, it still showed good profiling over the full 60 day period (gray/third graph from top). However, in contrast, no release of growth factors was observed for the preparation of PRP in PBS beyond the first 10 days (blue/first graph from top). Accordingly, it is evident that the composition devoid of the polymer lost any ability for sustained effect because of the dilution. However, very clearly, the polymer supports the sustained delivery of growth factors in both the compositions that had it. The growth factor release from the polymer validates the slow release of these proteins for long term availability and therapeutic efficacy.

### Example 11: Effect of Composition Comprising PRP or GFC and Thermoresponsive Polymer on Azoospermia

In order to test the therapeutic effect of the compositions of the present disclosure, mature fertile rats were treated with Busulfan, a chemotherapy drug, that causes infertility by azoospermia in rats. The rats received two doses of Busulfan (each 15 mg/kg) intraperitoneally (IP) with 14 days interval. Upon treatment, the compositions of the present disclosure were administered to the rats in the following batches:

Batch 1: PRP as prepared in example 1.

Batch 2: GFC as prepared in example 2.

Batch 3: Composition as prepared in example 6.

The rats received one dose of 800 μl of the composition via local injection in each testis.

For each batch, initial data (control) of the fertile rats was recorded, and compared with the data for rats treated with Busulfan (Busulfan), followed by data generated after administration of the compositions of the present disclosure (PRP/GFC/Composition). The data is as provided in the table 9 below. The data points recorded were for: number of spermatogonia, number of spermatocytes, number of spermatids (round) and the spermatozoa count.

TABLE 9

| | | | | Batch 3 |
|---|---|---|---|---|
| | | | Batch 2 | Composition of GFC |
| | | Batch 1 | GFC obtained from | obtained from the PRP of |
| | | PRP of the present | the PRP of the | the present disclosure and |
| Condition | Parameters | disclosure | present disclosure | thermoresponsive polymer |
| Azoospermia | No of Mature Rat | 30 | 30 | 30 |
| | Treatment | Control/Busulfan/PRP | Control/Busulfan/GFC | Control/Busulfan/Composition |
| | Spermatogonia × | 29.1/1.1/22 | 29.1/1.1/24 | 29.1/1.1/27.8 |
| | 10\hat{ }6 | | | |
| | Spermatocytes × | 119.8/5.3/110 | 119.8/5.3/92 | 119.8/5.3/110.7 |
| | 10\hat{ }6 | | | |
| | Spermatids | 274/3.2/228.2 | 274/3.2/248.2 | 274/3.2/268.2 |
| | (round) × 10\hat{ }6 | | | |
| | Spermatozoa | 8/2/7 | 8/2/6.8 | 8/2/7.2 |
| | count * 10\hat{ }6 | | | |

As can be seen, the PRP, GFC and the compositions of the present disclosure are clearly successful in reversing the damage caused by the Busulfan treatment. While each of them has a drastic effect on the treatment of azoospermia, some of them are able to reverse the infertility to a very close proximation as compared to the original levels. This shows the therapeutic efficacy of the compositions of the present disclosure.

### Example 12: Effect of Composition Comprising PRP or GFC and Thermoresponsive Polymer on Azoospermia

In order to test the therapeutic effect of the compositions of the present disclosure, the compositions of the present disclosure are administered via intratesticular injection in non-obstructive azoospermia patients (fertile men between 25-60 years old). The compositions as prepared in example 1 and 2 (along with PBSCs) of the present disclosure were administered to the patients as 0.5 ml injections in each testis.

The data points recorded were for: number of primary & secondary spermatocytes; and presence or absence of sperm and spermatogenesis, as provided in table 10 below.

TABLE 10

| | | Batch 2 - Treatment |
|---|---|---|
| | Batch 1 - Treatment | with composition of |
| Parameters | with PRP | GFC and PBSCs |
| Primary & secondary | 23 (46)/15 (33) | 50 (54.9)/17 (18.7) |
| spermatocytes | | |
| No sperm and no | 15 (30)/9(18) | 22 (22.4)/20 (22.0) |
| spermatogenesis | | |

As can be seen, primary and secondary spermatocytes were found more in patients administered both with growth factors along with stem cells (PBSCs). The similar observation were made with respect to spermatogenesis. Hence for gametogenesis both stem cell and growth factor support is needed for better clinical outcome.

### Example 13: Preparation of Kit of the Present Disclosure

A kit was prepared in accordance with the requirements of the present disclosure. The kit so prepared comprises of the following components:
a) G-CSF;
b) a RBC activating agent selected from a group comprising: heparin, collagen, a calcium salt, hyaluronic acid, polygeline, thrombin, gelatin, EDTA, sodium citrate, starch, and a combination thereof:
c) a thermoresponsive polymer; and
d) an instruction manual.

The kit was prepared in a manner so that it can be used for the following:
a) processing of whole blood for preparation of PRP of the present disclosure as per example 1 above;
b) processing of whole blood for preparation of GFC from the PRP of the present disclosure as per example 2 above;
c) processing of conventional PRP for preparation of GFC of the present disclosure as per example 2 above;
d) preparing of the therapeutic compositions of the present disclosure comprising PRP and thermosensitive polymer as per example 6 above;
e) preparing of the therapeutic compositions of the present disclosure comprising GFC and thermosensitive polymer as per example 7 above;
f) preparing of the therapeutic compositions of the present disclosure comprising PRP and thermosensitive polymer, and PBSCs as per example 8 above; and/or
g) preparing of the therapeutic compositions of the present disclosure comprising GFC and thermosensitive polymer, and PBSCs as per example 8 above.

In addition to the above 4 components, separate kits were also prepared to comprise one platelet activating agent selected from a group comprising collagen, a calcium salt, hyaluronic acid, and thrombin.

In all these kits, a blood collection container comprising an anti-coagulant was also provided.

All the kits so prepared herein additionally comprise an instruction manual each having steps for: processing of the whole blood for processing of whole blood for preparation of PRP of the present disclosure; processing of whole blood for preparation of GFC from the PRP of the present disclosure; processing of conventional PRP for preparation of GFC of the present disclosure; preparing of the therapeutic compositions of the present disclosure comprising PRP and thermosensitive polymer; and preparing of the therapeutic compositions of the present disclosure comprising GFC and thermosensitive polymer. The instructional manual also comprises steps for processing of PBSCs and inclusion on additional therapeutic agent during preparation of any of the said compositions.

Additional embodiments and features of the present disclosure will be apparent to one of ordinary skill in art based on the description provided herein. The embodiments herein provide various features and advantageous details thereof in the description. Descriptions of well-known/conventional methods and techniques are omitted so as to not unnecessarily obscure the embodiments herein.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments in this disclosure have been described in terms of preferred embodiments, those skilled in the art will recognize that the embodiments herein can be practiced with modification within the spirit and scope of the embodiments as described herein.

Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

While considerable emphasis has been placed herein on the particular features of this disclosure, it will be appreciated that various modifications can be made, and that many changes can be made in the preferred embodiments without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, Band C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, F, H; B, F, I; C, D, G, D, H; C, D, I; C, E, G; C, E, H; C, E, I; C, F, G, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring 25 back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to anyone of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all attached claims. To give a few examples, the combination of claims 6, 5, 4(b), 3 and 2 is clearly and unambiguously envisaged in view of the claim structure. The same applies for the combinations of claims 6, 35 5, 4(a), 3 and 2, and, to give a few further examples which are not limiting, the combination of claim 4(a) and 2 and the combination of claim 5, 4(a) and 2.

1. A therapeutic composition comprising a platelet rich plasma (PRP) or a growth factor concentrate derived therefrom and a thermoresponsive polymer.

2. The therapeutic composition of claim 1, wherein the PRP is a conventional PRP; or a PRP having a platelet count that is about 10 to 15-fold greater than starting whole blood sample from same subject, a red blood cell (RBC) count that is about 60 to 80-fold lower than starting whole blood sample from same subject, a white blood cell (WBC) count that is about 10 to 30-fold lower than starting whole blood sample from same subject, or any combination thereof.

3. The therapeutic composition of claim 1, wherein the growth factor concentrate comprises growth factor(s) selected from a group comprising VEGF, EGF, bFGF, IGF-1, PDGF-BB and TGF-b1 or any combination thereof.

4. The therapeutic composition of claim 3, wherein concentration of the VEGF ranges from about 500-1300 pg/mL, concentration of the EGF ranges from about 100-2000 pg/mL, concentration of the bFGF ranges from about 25-500 pg/mL, concentration of the IGF-1 ranges from about 500-1000 ng/mL, concentration of the PDGF-BB ranges from about 20-500 ng/mL, and concentration of the TGF-b1 ranges from about 100-2000 ng/mL.

5. The therapeutic composition claim 1, comprising peripheral blood stem cells (PBSCs), at a concentration ranging from about 10% to 50%.

6. The therapeutic composition of claim 1, wherein the PRP or the PBSCs is autologous; or derived from umbilical cord blood, bone marrow, fresh or expired platelet concentrates from blood banks, or buffy coat from blood banks.

7. The therapeutic composition of claim 1, comprising an additional therapeutic agent selected from a group comprising hormone, growth factor, protein, cell, cell secretome, and drug, or any combination thereof; and wherein the agent is selected from a group comprising follicle stimulating hormone (FSH), luteinizing hormone (LH), high-density lipoprotein (HDL), steroidogenic acute regulatory protein (StAR) and stem cell, or any combination thereof.

8. The therapeutic composition of claim 7, wherein the growth factor is selected from a group comprising VEGF, NGF, FGF, HGF, I-IGF-I, EGF, PDGF, Transforming growth factor-b/family, and SGF, or any combination thereof.

9. The therapeutic composition of claim 7, wherein concentration of the additional therapeutic agent ranges from about 20% to 30% of the composition; and wherein when the additional therapeutic agent is a growth factor, the concentration ranges from about 4-fold to 10-fold when compared to the physiological levels of constituting whole blood.

10. The therapeutic composition of claim 1, wherein the thermoresponsive polymer is selected from a group comprising a copolymer comprising poly(N-isopropylacrylamide-co-n-butyl methacrylate) and polyethylene glycol; copolymer comprising poly(ethylene oxide) (PEO) and poly(propylene oxide) (PPO), a NIPAM based polymer, amphiphilic block copolymers, ABA triblock copolymers and poloxamer, or any combination thereof; and wherein the thermoresponsive polymer exists in a liquid form at a temperature ranging from about −20° C. to +27° C., and in a gel form at a temperature ranging from about +27.1° C. to +60° C.

11. The therapeutic composition of claim 1, wherein concentration of the thermoresponsive polymer ranges from about 10% to 50%.

12. The therapeutic composition of claim 1, wherein concentration of the PRP or the growth factor concentrate ranges from about 10% to 90%.

13. The therapeutic composition of claim 1, wherein the PRP or the growth factor concentrate and the thermoresponsive polymer are present at a ratio ranging from about 90:10 to 10:90.

14. A method for preparing the therapeutic composition of claim 1, comprising mixing the PRP or the growth factor concentrate derived therefrom with the thermoresponsive polymer to obtain the composition.

15. The method of claim 14, wherein the PRP or the growth factor concentrate is mixed with the thermoresponsive polymer at a ratio ranging from about 57 90:10 to 10:90, to obtain the composition comprising about 10% to 90% of the PRP or the growth factor and about 10% to 50% of the thermoresponsive polymer.

16. The method of claim 14, wherein the thermoresponsive polymer is in a powder form or solution form while mixing with the PRP or the growth 5 factor concentrate; and wherein the solution comprises the polymer in water or saline.

17. The method of claim 14, comprising adding the peripheral blood stem cells or the additional therapeutic agent, or both, to the PRP or the GFC, and wherein the addition is carried out prior to mixing with the thermoresponsive polymer.

18. The method of claim 14, wherein the peripheral blood stem cells are added in the form of a solution, prepared by steps of:
incubating whole blood collected in an anti-coagulant container with a red blood cell (RBC) aggregating agent selected from the group consisting of: heparin, collagen, a calcium salt, hyaluronic acid, polygeline, thrombin, gelatin, EDTA, sodium citrate, starch, and a combination thereof;
subjecting the whole blood to centrifugation at a speed of about 1200 rpm for about 15 minutes;
removing top layer containing platelet-poor plasma and transferring middle buffy-coat layer containing PBSCs to another sterile tube;
subjecting the buffy coat layer to centrifugation at a speed of about 2000 rpm for about 10 minutes or filtration to separate PBSCs to obtain a solution comprising the PBSCs.

19. The method of claim 14, wherein the blood is subjected to administration of G-CSF at least one to three days prior to its withdrawal from a subject; and wherein the G-CSF enhances the WBCs in the blood by about 5-folds.

20. The method of claim 14, wherein the PRP is prepared by method comprising:
incubating whole blood with a red blood cell (RBC) aggregating agent selected from a group comprising: heparin, collagen, a calcium salt, hyaluronic acid, polygeline, thrombin, gelatin, EDTA, sodium citrate and starch, or any combination thereof;
subjecting the whole blood incubated with the RBC aggregating agent to a first centrifugation to obtain a supernatant containing platelets;
subjecting the supernatant to a second centrifugation to obtain a platelet pellet and platelet-poor plasma (PPP); and
resuspending the platelet pellet in PPP to obtain the PRP.

21-40. (canceled)
